(19) United States
(12) Patent Application Publication
(10) Pub. No.: US 2020/0031796 A1
(43) Pub. Date: Jan. 30, 2020

(54) COMPOSITION COMPRISING A COMPOUND OF THIOLACTONE TYPE AND A PARTICULAR SOLVENT AND PROCESS FOR TREATING KERATIN MATERIALS WITH THE COMPOSITION

(71) Applicant: L'Oreal, Paris (FR)

(72) Inventors: Sébastien VOISIN, Saint-Ouen (FR); Nicolas DAUBRESSE, Saint-Ouen (FR); Manon CHAUMONTET, Sain-Ouen (FR)

(21) Appl. No.: 16/469,223

(22) PCT Filed: Dec. 21, 2017
(86) PCT No.: PCT/EP2017/084073

§ 371 Date: Jun. 13, 2019

(30) Foreign Application Priority Data
Dec. 21, 2016 (FR) 1662989

(51) Int. Cl. C07D 331/04; C07D 333/38; C07D 333/32; C07D 335/02; A61K 8/49; A61Q 5/06

(52) U.S. Cl. CPC C07D 331/04; C07D 333/38; A61Q 5/06; C07D 335/02; A61K 8/4986; C07D 333/32

(57) ABSTRACT

The present invention relates to a composition comprising: (a) at least one compound of thiolactone type, and (b) one or more solvents, which may be identical or different, chosen from polar (non-)protic organic solvents; the pH of said composition being less than or equal to 7.

The present invention relates to a composition comprising at least one compound of thiolactone type and at least one particular solvent and to a process for treating keratin materials, in particular human keratin fibres, comprising the application of the composition, in particular for shaping keratin fibres, and in particular for straightening/relaxing the hair.

In the hair field, consumers wish to have available compositions which make it possible to introduce a temporary change to their head of hair, while targeting good shape retention of the effect produced. In general, it is desired for the change to withstand shampooing for at least fifteen days or even more, depending on the nature of said change.

Heat treatments are generally used to modify the shape of the head of hair in a long-lasting manner. These treatments allow a visual modification of the appearance of the hairstyle, combining a decrease in the degree of frizziness, a reduction in overall volume of the head of hair, a decrease in little curls, a gain in manageability, a straighter overall appearance, a substantial gain in sheen, and a resistance to humidity and to heat in order to maintain the hairstyle throughout the day.

Moreover, this type of treatment has the advantage of facilitating the daily maintenance of the head of hair, with the use of fewer care products, in particular rinse-out care products such as hair conditioners or masks, or leave-in care products such as sera, care creams or balms, or taming mousses. Drying of the hair is facilitated, with a much shortened blow-drying time and a decrease in the daily use of flat irons, in terms both of time and intensity. This thus makes it possible to limit the risks of damaging the hair through combined factors of mechanical and thermal stress.

Several techniques are combined with these heat treatments. A first technique is based on the use of compositions based on thiol-based reducing agents. These techniques require strict adherence to the application conditions recommended by the suppliers, in particular in terms of amount and leave-in time. In addition, they may be contraindicated on hair that is too sensitized and may not be compatible with same-day application of other treatments, such as dyeing or bleaching operations. Moreover, they have an unpleasant smell.

Another technique is based on the use of compositions based on formol (or formaldehyde) and derivatives thereof. These treatments have the particular feature of being robust, perfectly compatible with all the other conventional hair treatments, such as the thiol-based straightening operations previously mentioned, alkaline relaxing operations, dyeing or bleaching operations of all types, performed before or after. They provide the hair with excellent manageability, a very bright sheen and easy daily care. However, in the event of repeated applications, further damage to the hair occurs, which can lead to breaking of the hairs. Furthermore, for toxicological reasons, the use of some of these compounds is now prohibited and/or regulated. It is therefore increasingly sought to avoid the use of such substances, which may prove to be aggressive to the hair and other keratin materials.

Another technique is based on the use of compositions based on acids, and most particularly on the use of glyoxylic acid. Patent application WO 2011/104 282 thus proposed a novel process for semi-permanently straightening the hair, which consists in applying an α-keto acid solution to the hair for 15 to 120 minutes, then drying and, finally, straightening the head of hair with an iron at a temperature of about 200° C. The α-keto acid employed is preferably glyoxylic acid. However, it has been noted that glyoxylic acid may not be well tolerated, in particular when the scalp is sensitive and/or irritated. Its volatility, amplified by the use of heat from the iron, can also be a problem. Moreover, the prior art compositions can modify the hair and/or modify the colour thereof, and most particularly can cause unwanted changes in artificial colourings such as oxidation colours.

Treatments using a composition comprising a base combined with a heat treatment have also been proposed for straightening the hair. Such treatments make it possible to obtain good relaxing of curls, but can lead to impairment of the hair fibre. EP 1837010 especially describes a straightening/relaxing process using a composition comprising sodium hydroxide and a heat treatment. WO 2007/144707 describes a straightening/relaxing process using a composition comprising a non-hydroxylated base such as monoethanolamine or ethylenediamine, combined with a heat treatment. WO 2009/117344 also describes a straightening/relaxing process using a composition comprising a non-hydroxylated base and a protein-denaturing agent, combined with a heat treatment. In order to limit hair fibre impairment, it has also been proposed to use compositions comprising weak acids at alkaline pH, combined with a heat treatment. WO 2010/049434 describes, for example, a straightening/relaxing process in which a composition comprising a dicarboxylic acid, such as maleic acid, and a heat treatment are applied.

There is still a need to develop a process for treating keratin materials, in particular keratin fibres such as the hair, more particularly a process for shaping the hair, in particular straightening the hair, in a way that is efficient and long-lasting, while at the same time limiting degradation of the hair, in particular in a way that is persistent with respect to successive shampooing operations.

The applicant has now discovered that the use of a composition comprising at least one compound of thiolactone type and at least one particular solvent makes it possible to achieve the desired long-lasting shaping properties.

Thus, a subject of the present invention is a composition comprising:
(a) one or more compound(s) chosen from the compounds of formula (I) below:

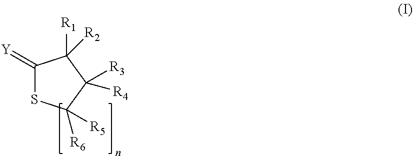

and also the organic or mineral acid or base salts thereof, the optical isomers thereof, the geometrical isomers thereof, the tautomers thereof, and the solvates thereof such as hydrates;
in which formula (I):
   Y represents an oxygen or sulfur atom ora group NR with R representing a hydrogen atom or a (C₁-C₆)alkyl or hydroxy(C₁-C₆)alkyl group; preferably, Y represents an oxygen atom;
   R₁, R₂, R₃, R₄, R₅ and R₆, which may be identical or different, represent:
      i) a hydrogen atom,
      ii) hydroxyl,
      iii) amino —NR′R″, with R′ and R″, which may be identical or different, representing a hydrogen atom or a (C₁-C₆)alkyl or hydroxy(C₁-C₆)alkyl group;
      iv) cyano,
      v) —(Y′)ₚ—C(Y″)—(Y′″)_{q}—R₇ with R₇ representing a hydrogen atom or a (C₁-C₆)alkyl group, p and q, which may be identical or different, being equal to 0 or 1, preferably with p+q=0 or 1, Y′, Y″ and Y′″, which may be identical or different, representing an oxygen or sulfur atom, or NR with R as defined previously, preferably Y′, Y″ and Y′″ represent an oxygen atom;
      vi) optionally substituted (C₁-C₆)alkyl,
      vii) optionally substituted (C₂-C₆)alkenyl,
      viii) optionally substituted (C₁-C₆)alkoxy,
      preferably, said alkyl, alkenyl or alkoxy groups being optionally substituted with one or more groups chosen from hydroxyl and —(O)ₜ—C(O)—R₈ with t being equal to 0 or 1, R₈ represents a) a hydrogen atom, b) a hydroxyl group, c) (C₁-C₆)alkyl, d) (C₁-C₆)alkoxy, e) (di)(C₁-C₆)(alkyl)amino, f) (di)hydroxy(C₁-C₆)alkylamino;
      or alternatively:
      ix) R₁ and R₃ together form a covalent bond;
      x) R₁ and R₂, and/or R₃ and R₄, and/or R₅ and R₆ form, together with the carbon atom that bears them, a double bond oxo ═O or ═C(R₉)—R₁₀ with R₉ and R₁₀ representing a) a hydrogen atom or a group chosen from b) hydroxyl, c) (C₁-C₆)alkyl optionally substituted with one or more hydroxyl groups, and d) (C₂-C₆)alkenyl, e) —(Y′)ₚ—C(Y″)—(Y′″)_{q}—R₇ with Y′, Y″, Y′″, p, q and R₇ being as defined previously;
   n represents an integer between 0 and 6 inclusive, particularly between 0 and 4 inclusive, more particularly between 0 and 3 inclusive, preferably n is 1 or 2, and more preferentially n is 1; it being understood that:
      when n is greater than or equal to 2, then the contiguous groups C(R₅)—R₆ may be identical or different; and
   (b) one or more solvents, which may be identical or different, chosen from polar (non-)protic organic solvents;
   the pH of said composition being less than or equal to 7.

Another subject of the invention is a process for treating keratin materials, in particular keratin fibres, especially human keratin fibres such as the hair, for shaping keratin fibres, in particular straightening and/or relaxing said fibres, using the composition as defined previously.

The composition of the invention makes it possible in particular to obtain good long-lasting relaxation of curls, in particular with respect to shampooing operations, while limiting the degradation of the hair. It also makes it possible to limit the formation of frizz in the presence of moisture.

Other characteristics and advantages of the invention will emerge more clearly on reading the description and the examples that follow.

In the following text, unless indicated otherwise:
the term “organic or mineral acid salt” is intended to mean cosmetically acceptable organic or mineral acid salts, more particularly the salts chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)OH; xiv) triflic acid CF₃SO₃H; and xv) tetrafluoroboric acid HBF₄;
the term “alkyl” is intended to mean a linear or branched hydrocarbon-based radical containing from 1 to 8 carbon atoms, in particular from 1 to 6 carbon atoms, preferably from 1 to 4 carbon atoms, for example methyl, ethyl, n-propyl, isopropyl, butyl, n-pentyl, n-hexyl, preferably methyl;
the term “alkenyl” is intended to mean a linear or branched hydrocarbon-based radical containing from 2 to 8 carbon atoms, in particular from 2 to 6 carbon atoms, preferably from 2 to 4 carbon atoms, and comprising at least one or more conjugated or non-conjugated unsaturations, such as ethylenyl, n-propylenyl, isopropylenyl, butylenyl, n-pentylenyl, n-hexylenyl;
the term “alkoxy” is intended to mean an alkyl oxy group with “alkyl” or “alkenyl” as defined previously;
the term “optionally substituted” preceding alkyl, alkenyl or alkoxy groups is intended to mean that said groups may be substituted on the hydrocarbon-based radical with one or more groups, which may be identical or different, chosen from i) hydroxyl, ii) thiol, iii) halogen, iv) (C₁-C₄)alkoxy, v) hydroxy(C₂-C₄)alkoxy; vi) (di)hydroxy(C₁-C₄)(alkyl)amino, vii) cyano, viii) nitro(so), ix) Rₐ—Zₐ—C(Z_{b})—Z_{c}— and x) Rₐ—Zₐ—S(O)ₜ—Z_{c}— with Z_{b} representing an oxygen or sulfur atom or a group NRₐ′, Zₐ and Z_{c}, which may be identical or different, representing a bond, an oxygen or sulfur atom or a group NRₐ; Rₐ representing a hydrogen atom or a (C₁-C₄)alkyl group and R_{a′} representing a hydrogen atom or an alkyl group and t being 1 or 2;
moreover, the addition salts that may be used in the context of the invention are especially chosen from salts of addition with a cosmetically acceptable base such as the basifying agents as defined below, for instance alkali metal hydroxides such as sodium hydroxide or potassium hydroxide, aqueous ammonia, amines or alkanolamines;
the term “relaxing” includes, according to the invention, the relaxing, straightening or uncurling of Caucasian or African hair. The term “to relax” includes, according to the invention, the act of relaxing, straightening or uncurling of Caucasian or African hair;
the expression “at least one” is equivalent to “one or more”; and
the expression “inclusive” for a range of concentrations means that the limits of the range are included in the defined interval.

The composition according to the invention comprises one or more compound(s) of formula (I).

According to one particular embodiment of the invention, the compound(s) of formula (I) are such that n is 1 or 2, and preferably of formula (Ia):

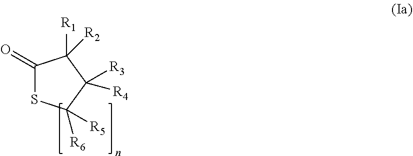

and also the organic or mineral acid or base salts thereof, the optical isomers thereof, the geometrical isomers thereof, the tautomers thereof, and the solvates thereof such as hydrates;
in which formula (Ia):
R₁, R₂, R₃, R₄, R₅ and R₆, which may be identical or different, represent:
   i) a hydrogen atom,
   ii) hydroxyl,
   iii) cyano,
   iv) amino —NR′R″, R′ and R″, which may be identical or different, representing a hydrogen atom or a (C₁-C₆)alkyl or hydroxy(C₁-C₆)alkyl group;
   v) —(Y¹)ₚ—C(O)—(Y″)_{q}—R₇ with R₇ representing a hydrogen atom or a (C₁-C₆)alkyl group, p and q, which may be identical or different, being equal to 0 or 1, with Y′ and Y″, which may be identical or different, representing an oxygen atom or NR with R as defined previously, preferably with p+q=0 or 1,
   vi) (C₁-C₆)alkyl optionally substituted with one or more groups chosen from hydroxyl and —C(O)—R₈ with R₈ representing a) a hydrogen atom, b) a hydroxyl group, c) (C₁-C₆)alkoxy,
   vii) (C₂-C₆)alkenyl,
   viii) (C₁-C₆)alkoxy,
      or alternatively:
   ix) R₁ and R₂, and/or R₃ and R₄, and/or R₅ and R₆ form, together with the carbon atom that bears them, a double bond oxo ═O or ═C(R₉)—R₁₀ with R₉ and R₁₀ representing a) a hydrogen atom or a group chosen from b) hydroxyl, c) (C₁-C₆)alkyl.

According to a particular embodiment of the invention, the compound(s) of formula (I) are such that n is 1, and preferably of formula (I′a):

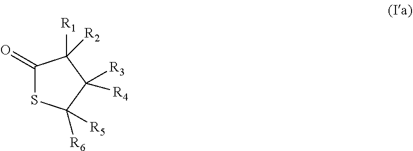

and also the organic or mineral acid or base salts thereof, the optical isomers thereof, the geometrical isomers thereof, the tautomers thereof, and the solvates thereof such as hydrates;
in which formula (I′a):
R₁, R₂, R₃, R₄, R₅ and R₆, which may be identical or different, are as defined previously, and in particular represent:
   i) a hydrogen atom,
   ii) hydroxyl,
   iii) cyano,
   iv) amino —NR′R″, R′ and R″, which may be identical or different, representing a hydrogen atom or a (C₁-C₆)alkyl or hydroxy(C₁-C₆)alkyl group;
   v) —(Y′)ₚ—C(O)—(O)_{q}—R₇ with R₇ representing a hydrogen atom or a (C₁-C₆)alkyl group, p and q, which may be identical or different, being equal to 0 or 1, with Y′ representing an oxygen atom or NR with R as defined previously, preferably with p+q=0 or 1, vi) optionally substituted (C₁-C₆)alkyl,
   vii) (C₂-C₆)alkenyl,
   viii) (C₁-C₆)alkoxy,
      said alkyl groups being optionally substituted with one or more groups chosen from hydroxyl and —(O)ₜ—C(O)—R₈ with t being equal to 0 or 1, R₈ representing a) a hydrogen atom, b) a hydroxyl group, c) (C₁-C₆)alkyl, d) (C₁-C₆)alkoxy, e) (di)(C₁-C₆)(alkyl)amino, f) (di)hydroxy(C₁-C₆)alkylamino;
      or alternatively:
   ix) R₁ and R₂, and/or R₃ and R₄, and/or R₅ and R₆ form, together with the carbon atom that bears them, a double bond oxo ═O or ═C(R₉)—R₁₀ with R₉ and R₁₀ representing i) a hydrogen atom or a group chosen from ii) hydroxyl, iii) (C₁-C₆)alkyl optionally substituted with one or more hydroxyl groups, in particular, R₃ and R₄ form, together with the oxygen atom, an oxo group and/or in particular, R₁ and R₂ represent, together with the carbon atom, ═C(R₉)—R₁₀.

According to one particular embodiment of the invention, the compound(s) of formula (I) are such that n is 1 or 2, and is of formula (Ib):

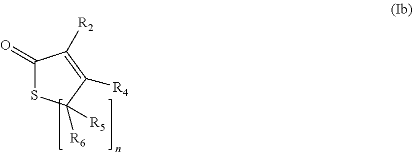

and also the organic or mineral acid or base salts thereof, the optical isomers thereof, the geometrical isomers thereof, the tautomers thereof, and the solvates thereof such as hydrates;
in which formula (Ib):
R₂, R₄, R₅ and R₆, which may be identical or different, represent:
   i) a hydrogen atom,
   ii) hydroxyl,
   iii) cyano,
   iv) amino —NR′R″, R′ and R″, which may be identical or different, representing a hydrogen atom or a (C₁-C₆)alkyl or hydroxy(C₁-C₆)alkyl group;
   v) —(Y′)ₚ—C(O)—(O)_{q}—R₇ with R₇ representing a hydrogen atom or a (C₁-C₆)alkyl group, p and q, which may be identical or different, being equal to 0 or 1, with p+q=0 or 1, with Y′ representing an oxygen atom or NR with R as defined previously,
   vi) (C₁-C₆)alkyl optionally substituted with one or more groups chosen from hydroxyl and —C(O)—R₈ with R₈ representing a) a hydrogen atom, b) a hydroxyl group, c) (C₁-C₆)alkoxy,
   vii) (C₂-C₆)alkenyl,
   viii) (C₁-C₆)alkoxy,
   or alternatively:
   ix) R₅ and R₆ form, together with the carbon atom that bears them, a double bond oxo ═O or ═C(R₉)—R₁₀ with R₉ and R₁₀ representing a hydrogen atom or a (C₁-C₆)alkyl group.

According to one particular embodiment of the invention, the compound(s) of formula (I) are of formula (Ic):

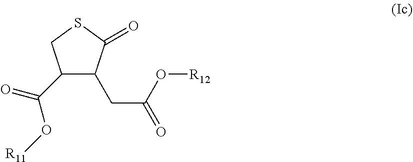

and also the organic or mineral acid or base salts thereof, the optical isomers thereof, the geometrical isomers thereof, the tautomers thereof, and the solvates thereof such as hydrates;
in which formula (Ic):
R₁₁ and R₁₂, which may be identical or different, represent a hydrogen atom or a (C₁-C₆)alkyl group, such as methyl or ethyl. More preferentially, R₁₁ and R₁₂ represent a hydrogen atom.

According to one particular embodiment, the compounds of formula (I′c) are chosen from:

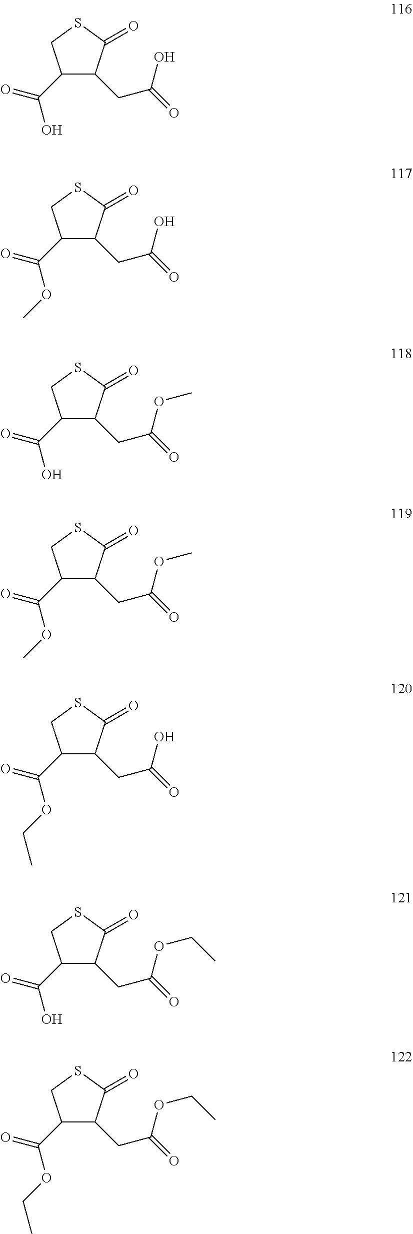

and also the organic or mineral base salts, the optical isomers, and the solvates such as hydrates.

According to a more particular embodiment of the invention, the compound(s) of formula (I) are of formula (I′a) or (Ib) and n is 1.

More preferentially, the compounds of formula (I) of the invention are chosen from the following compounds:

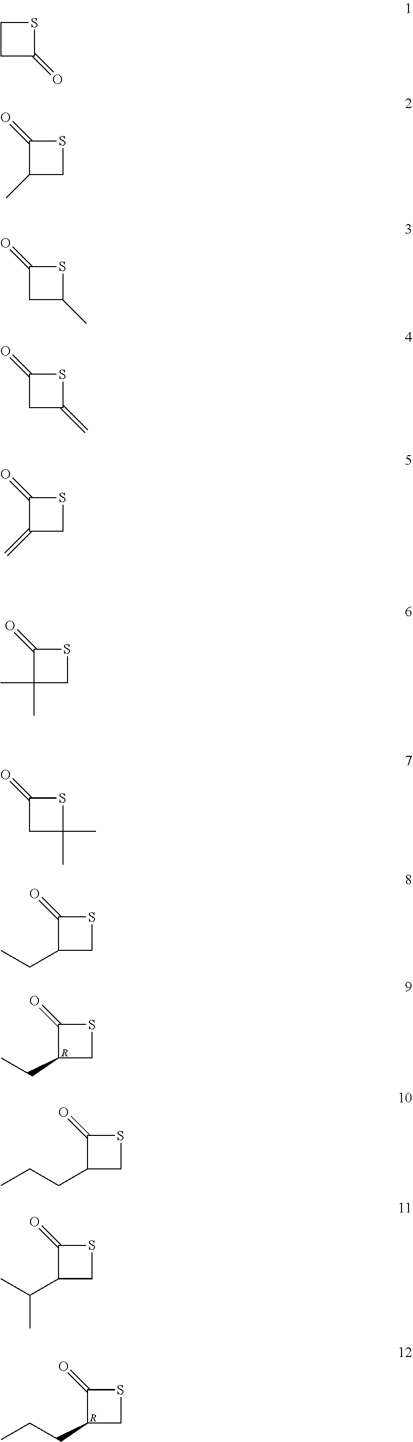

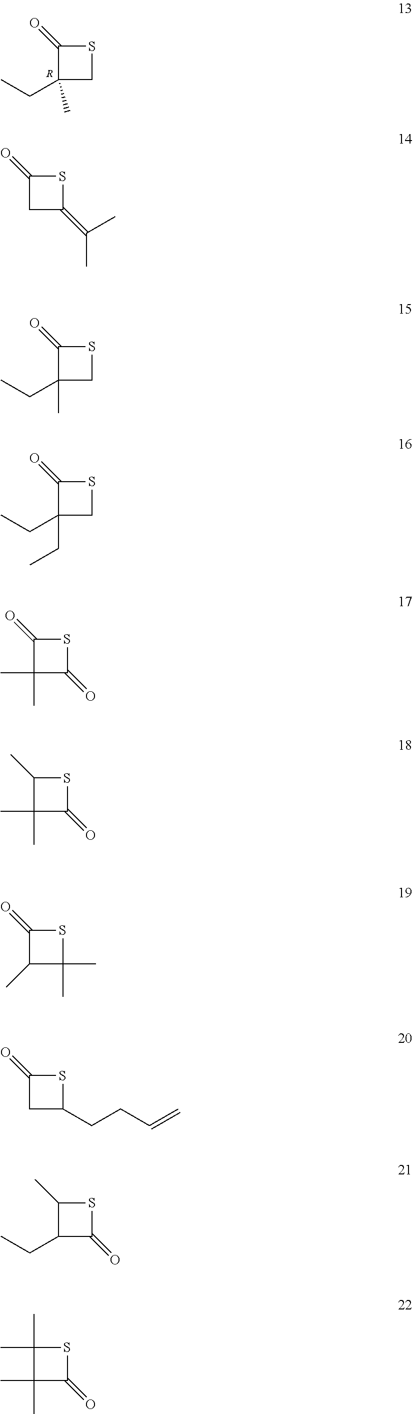

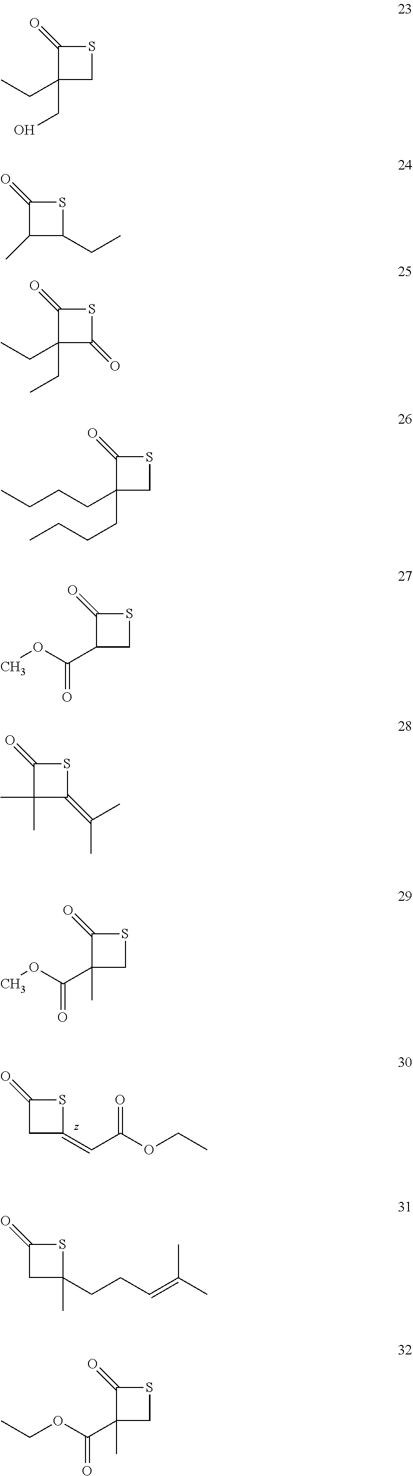

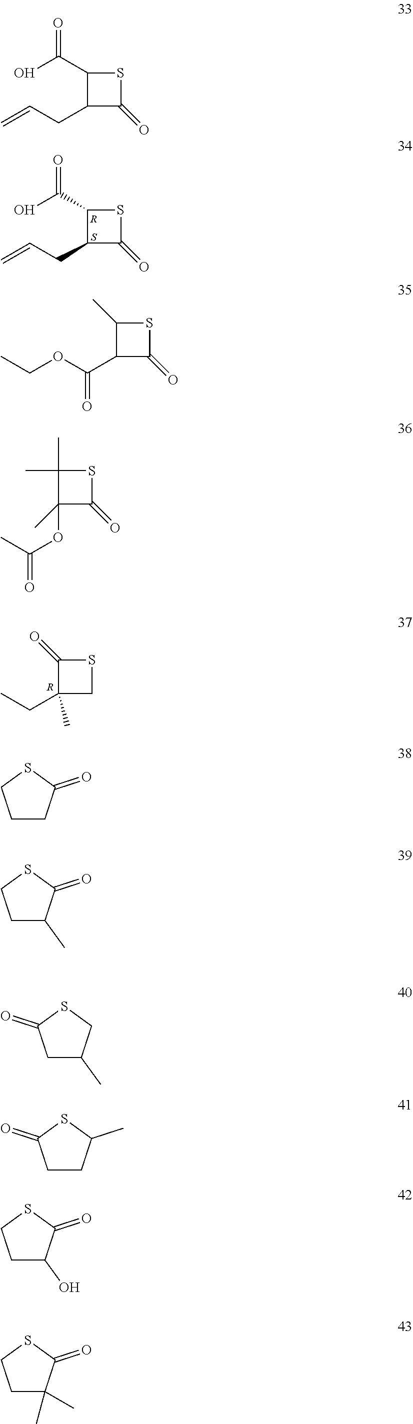

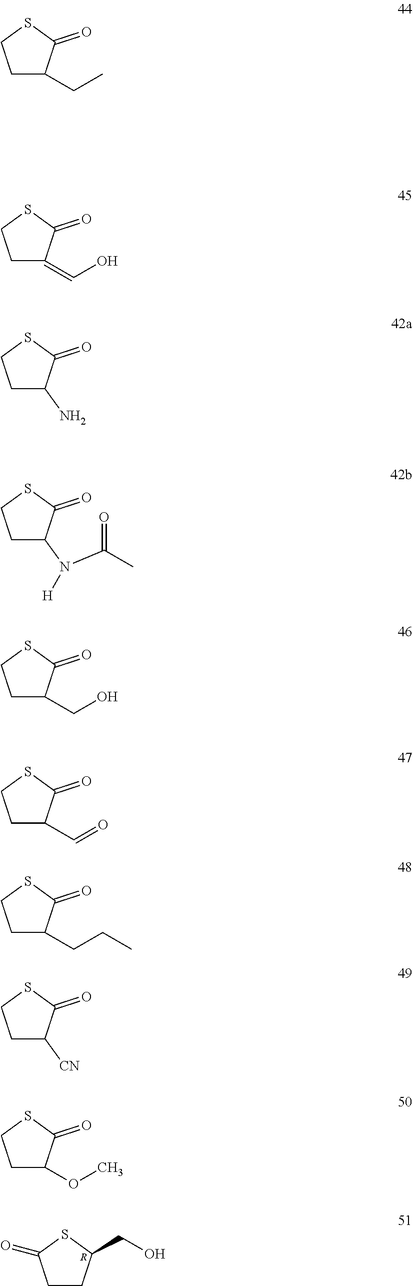

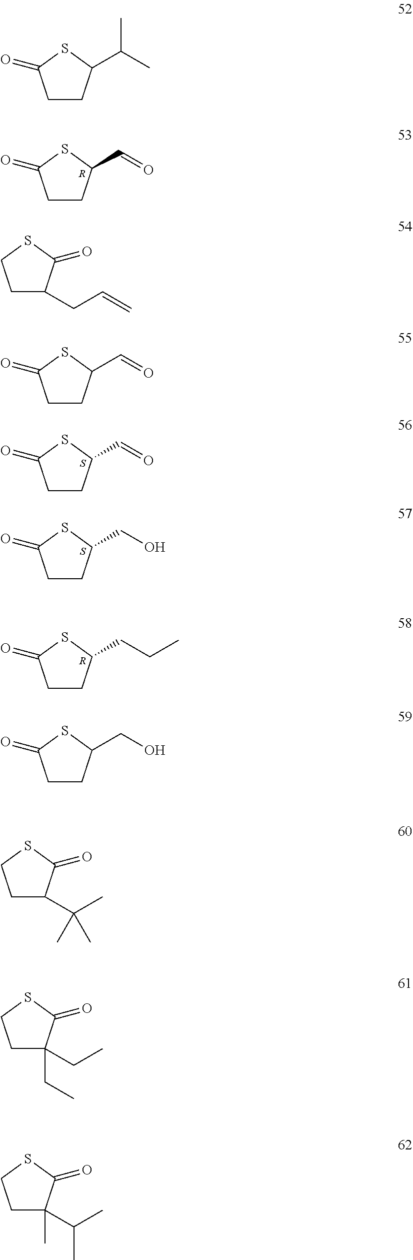

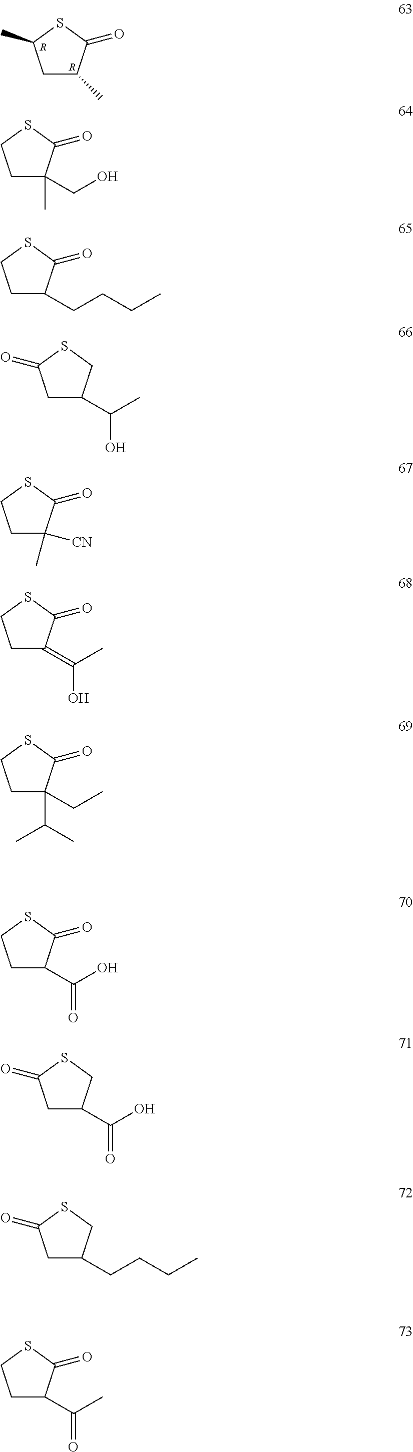

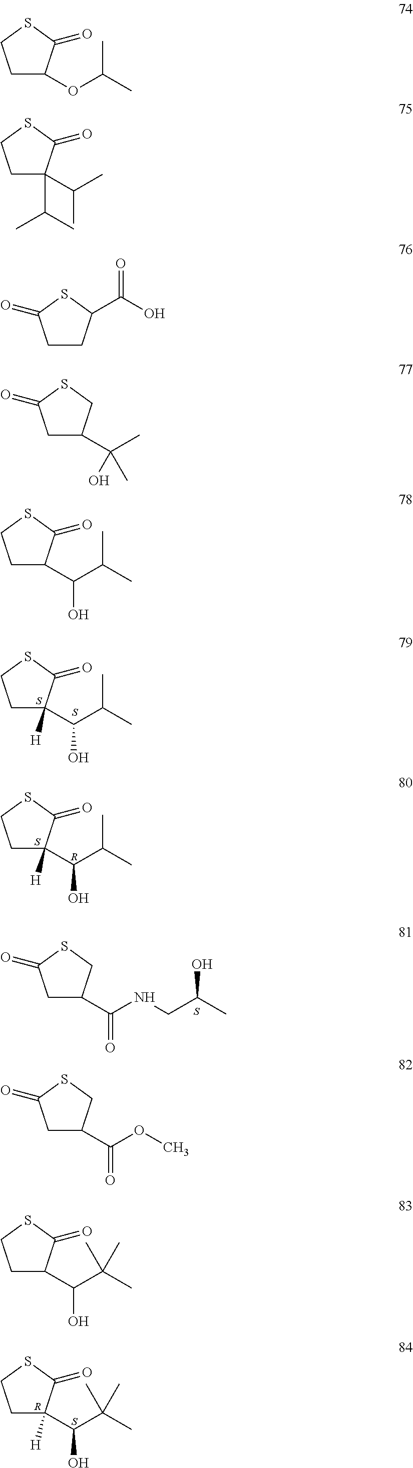

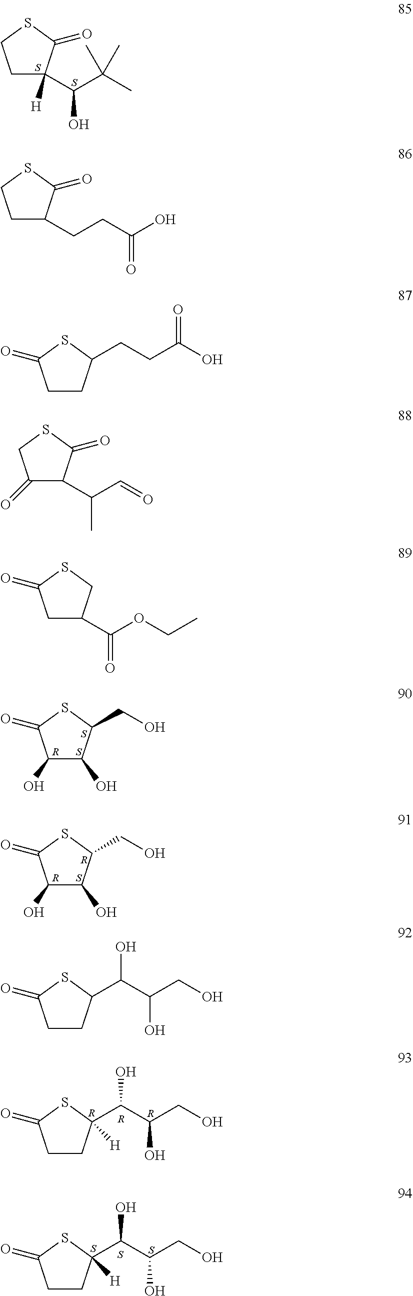

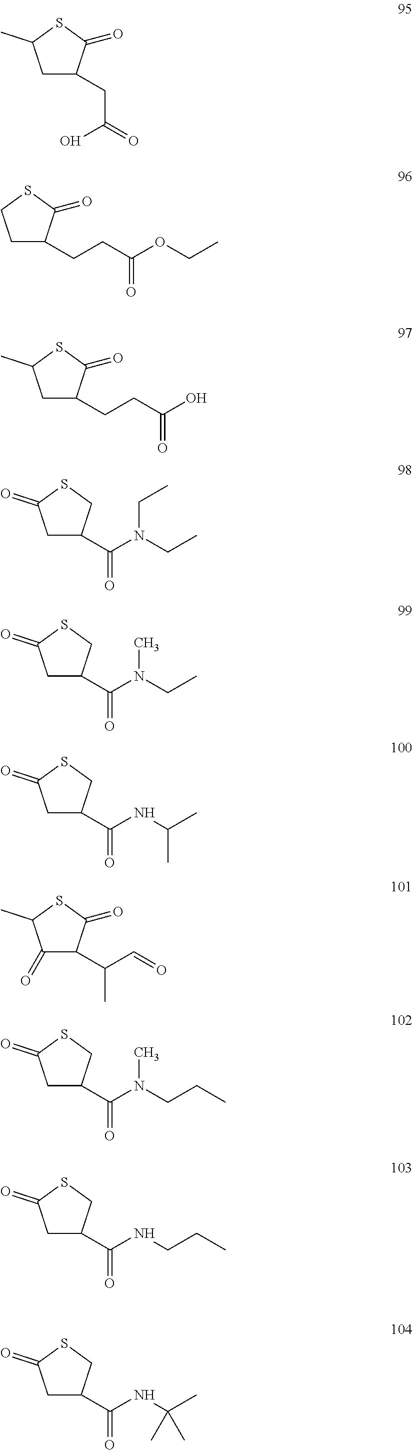

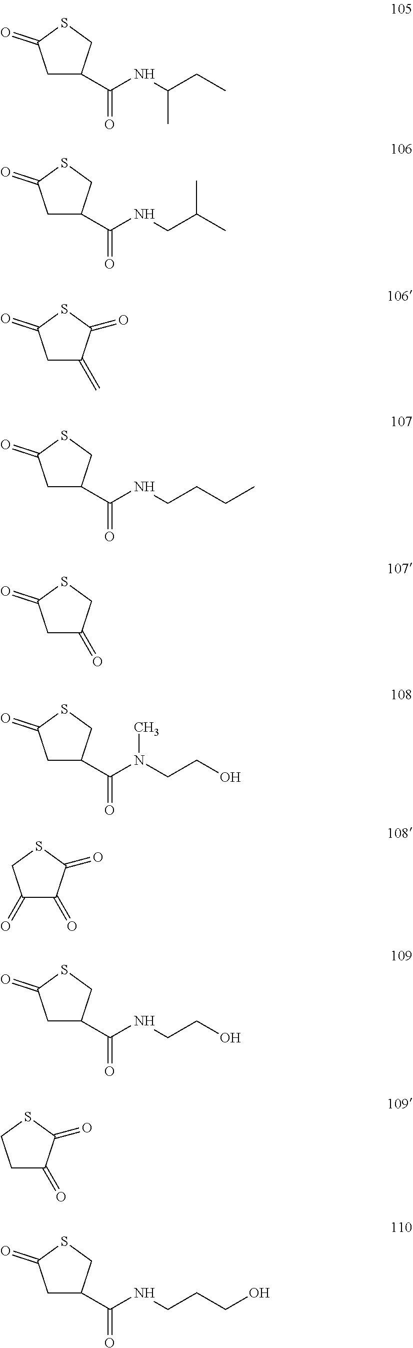

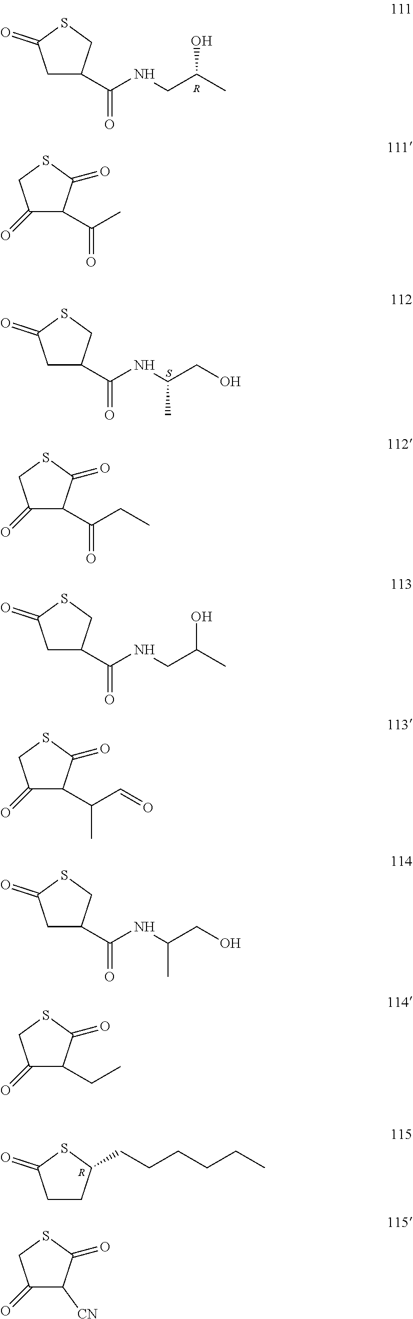

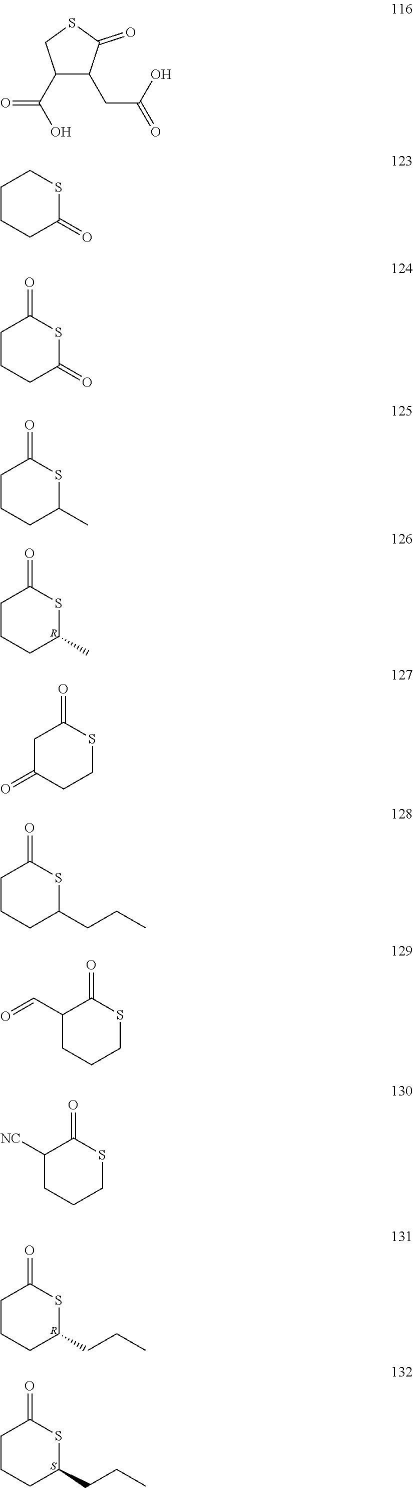

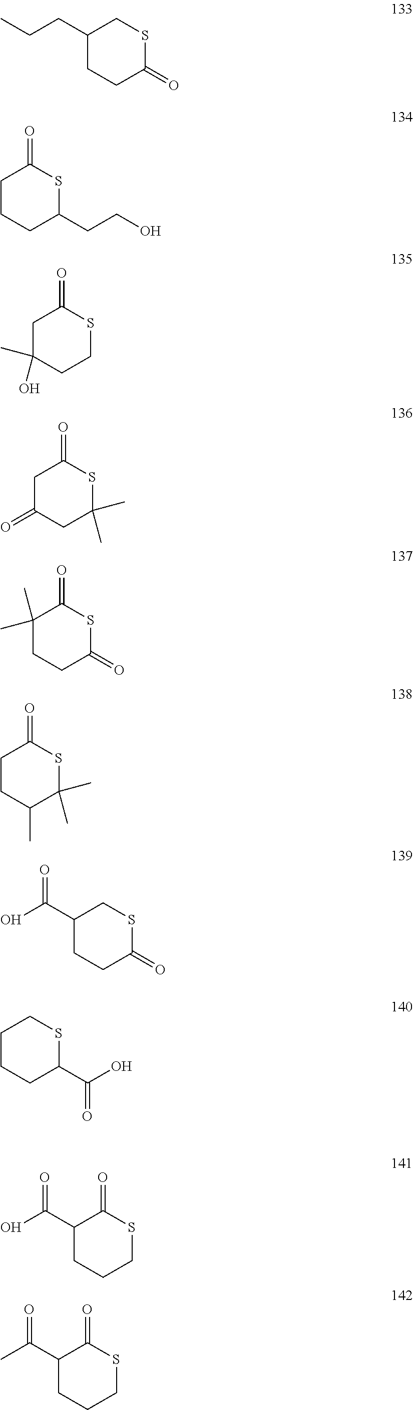

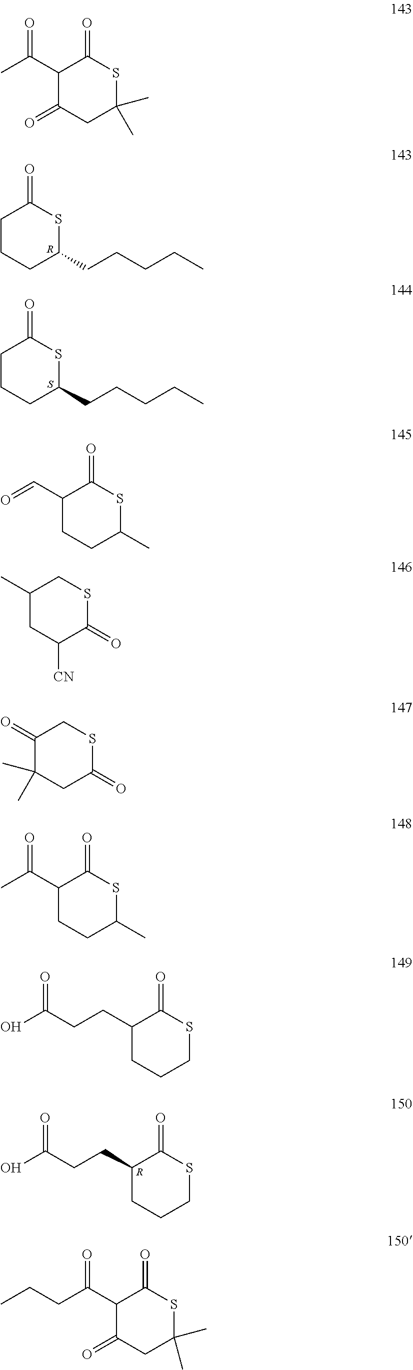

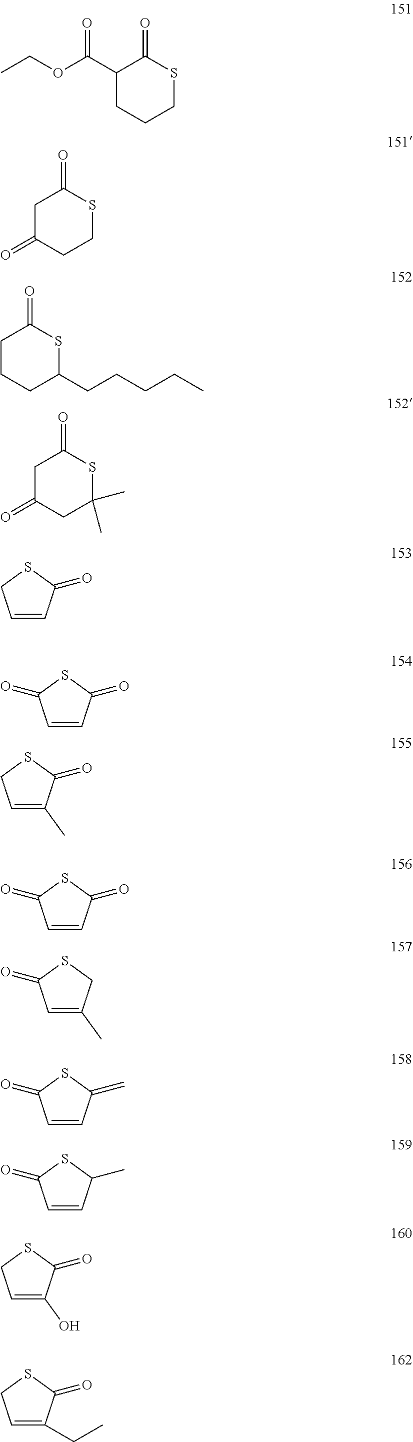

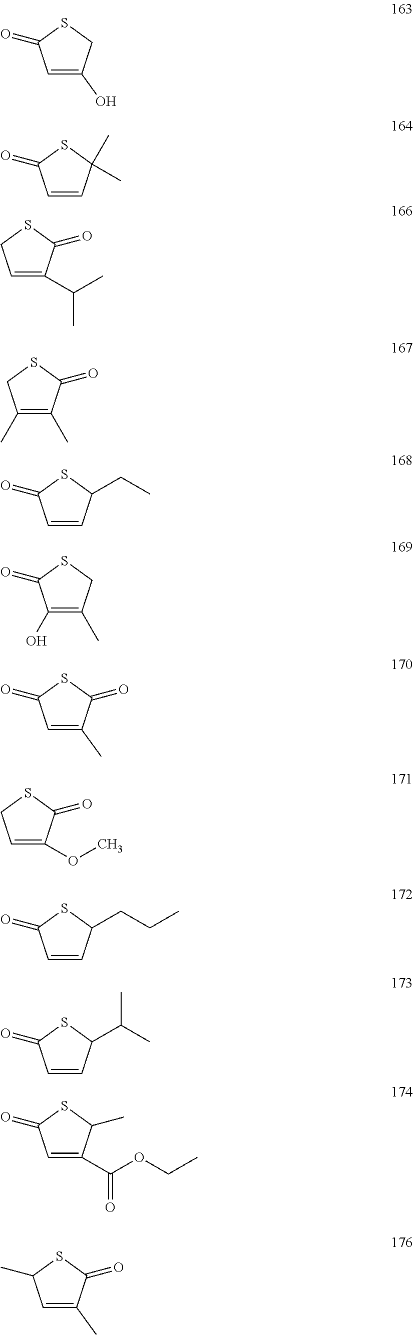

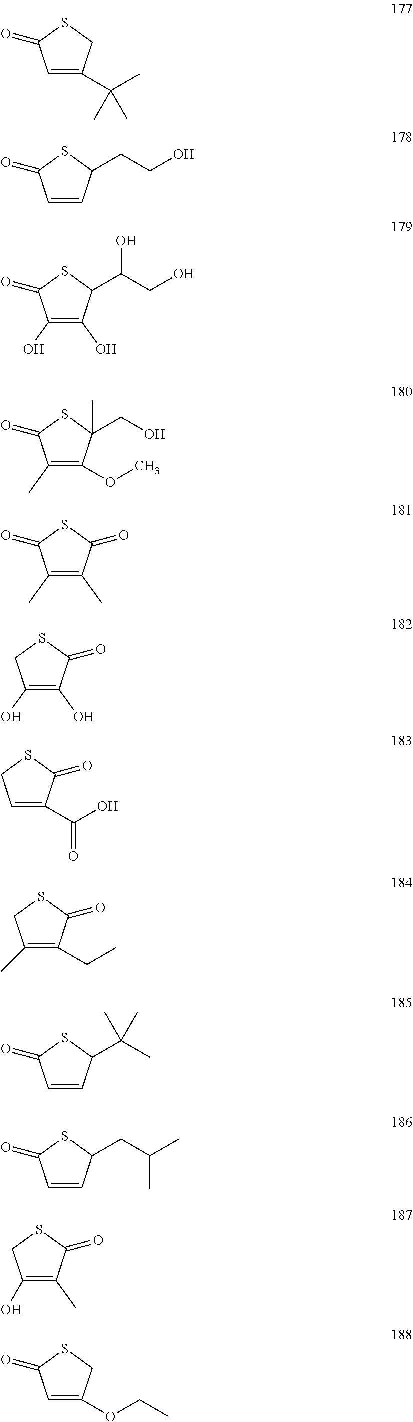

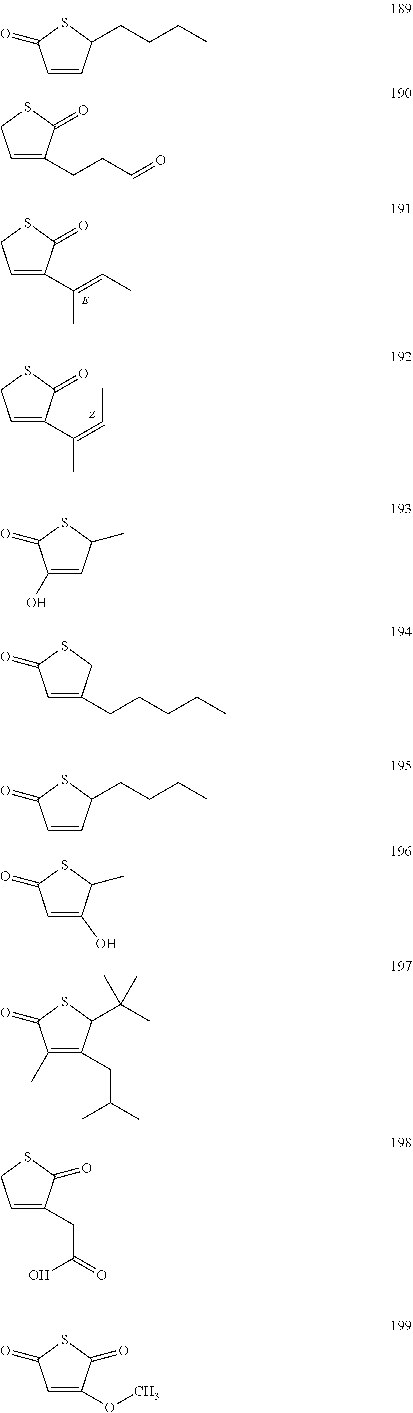

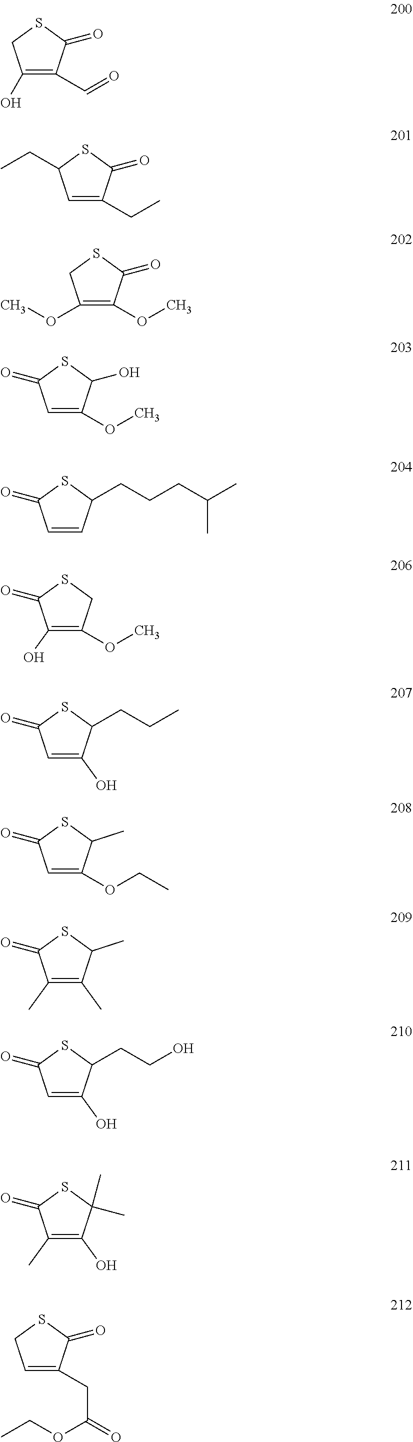

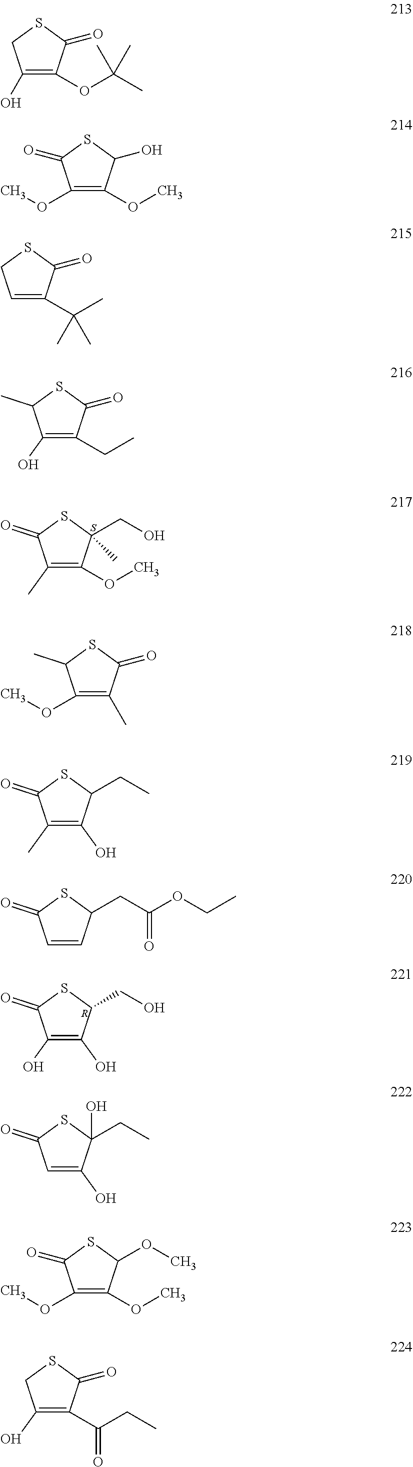

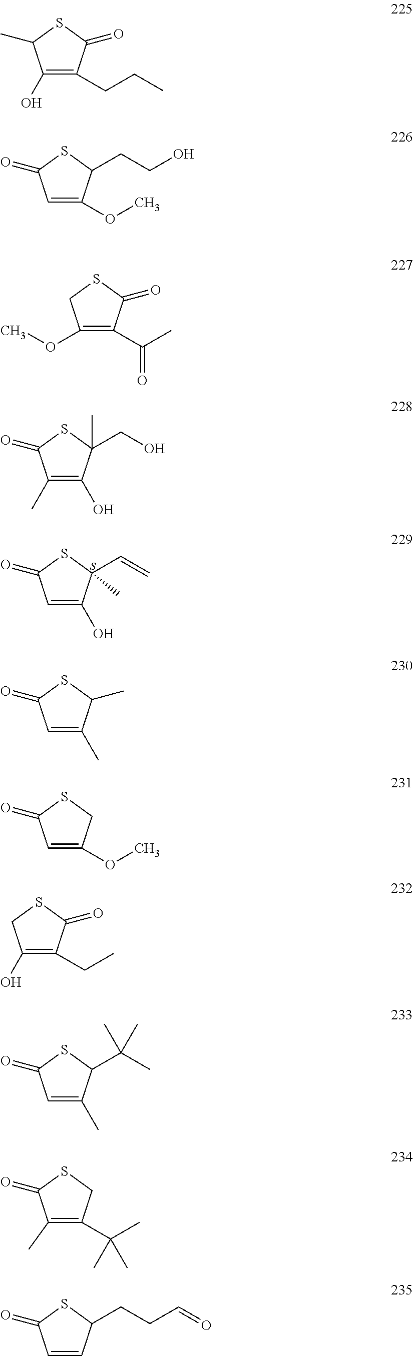

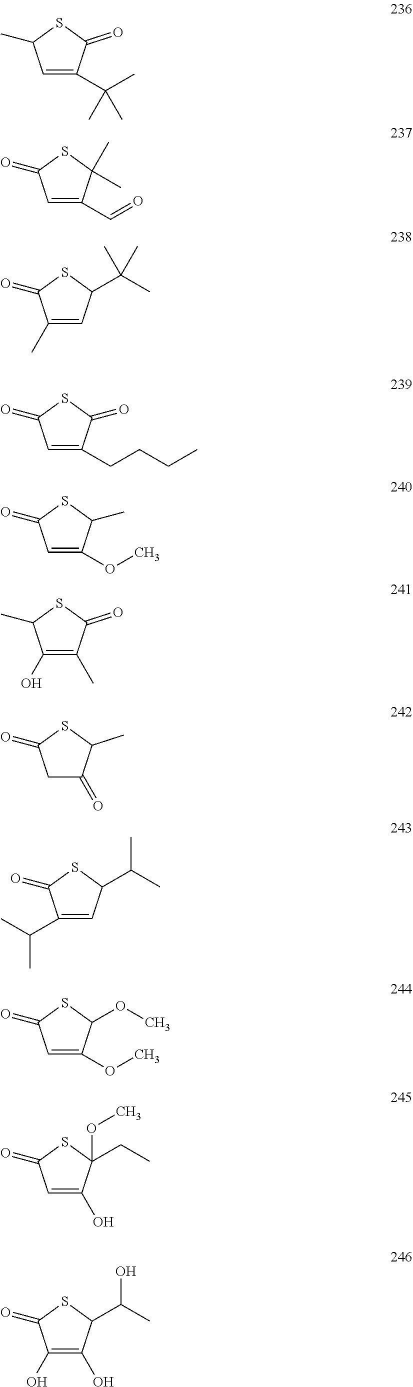

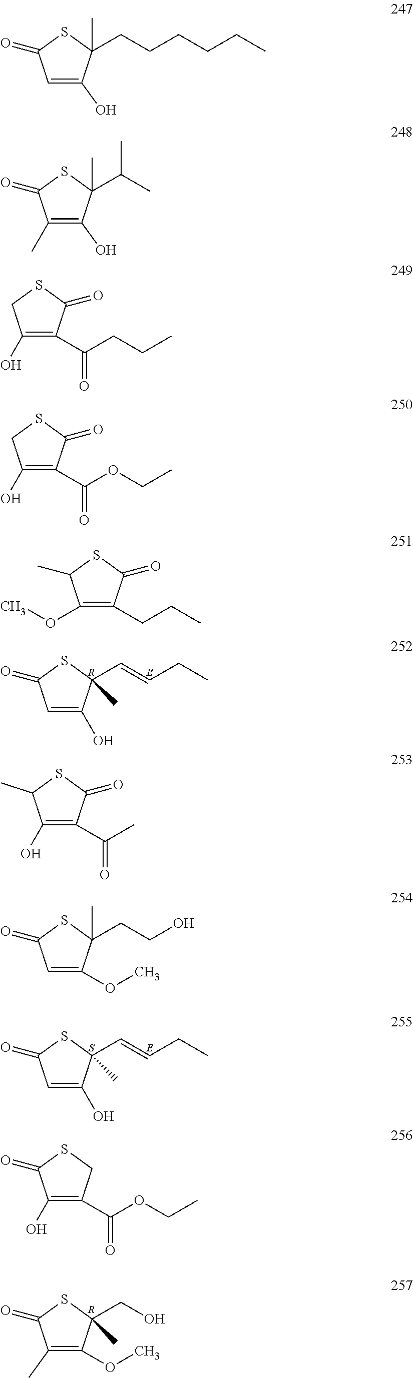

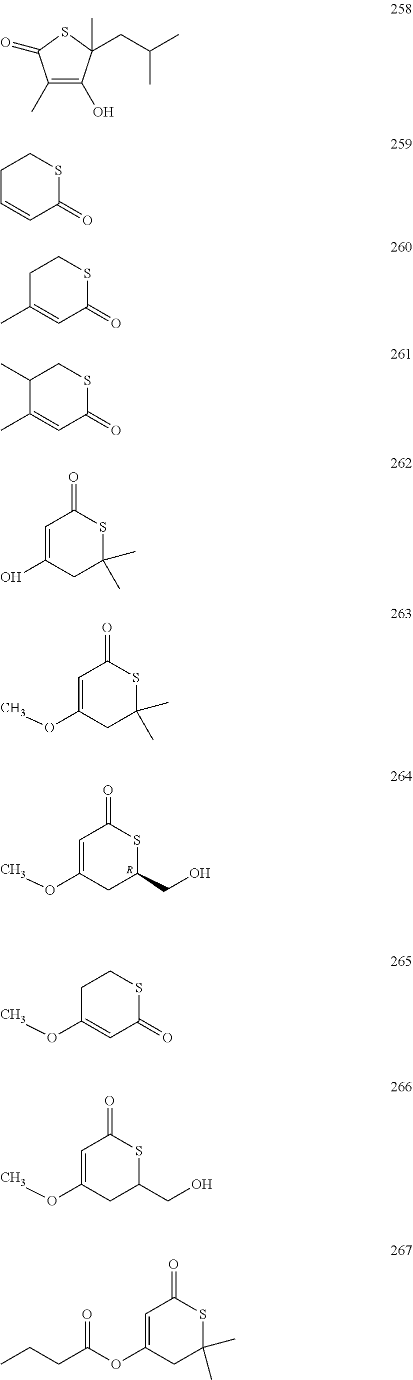

and also the organic or mineral acid or base salts thereof, the optical isomers thereof, the geometrical isomers thereof, the tautomers thereof, and the solvates thereof such as hydrates.

Preferably, the compound(s) of formula (I) is (are) chosen from the compounds of formulae (38) to (122) and (153) to (258), preferably from the compounds of formulae (38) to (42b), (70) to (71), (82), (86) to (87), (89), (90) to (95), (107′), (153) to (163), more preferably from the compounds of formulae (38), (42a), (42b), (71), (82), (89), (107′), (153), (163).

The process for preparing the compound of formula (I′b) is defined according to the following synthesis scheme:

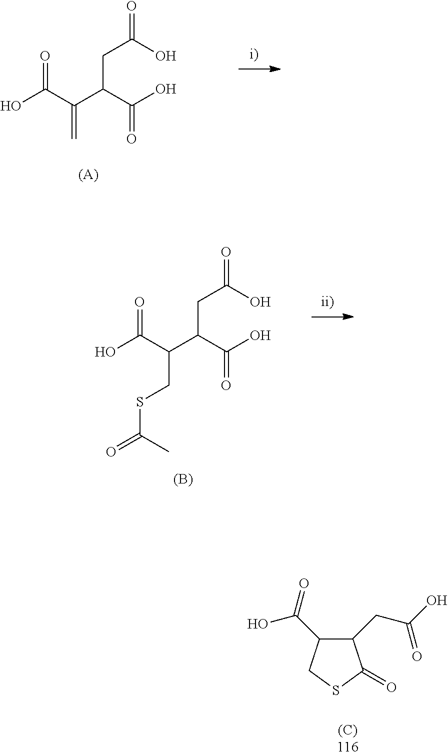

which consists in reacting, in step i), the 3-butene-1,2,3-tricarboxylic acid (A) with at least one molar equivalent of thioacetic acid, in particular in a non-protic organic solvent, which is preferably halogenated, such as dichloromethane, or ether of THF type, more particularly by heating the reaction medium up to the reflux of the solvent, preferably at a temperature of between 40° C. and 80° C., so as to give the compound (B); in step ii), the compound (B) reacts with an organic or inorganic strong acid such as hydrochloric acid, in particular in a polar protic solvent such as water, more particularly by heating at the reflux of the solvent, preferably at a temperature between 60 and 90° C., so as to give the thiolactone (C).

According to a particular embodiment, the composition comprises one or more compound(s) of formula (I) as defined previously, in an amount inclusively between 0.01% and 50%, in particular between 0.1% and 30%, more particularly between 1% and 20%, preferentially between 2% and 15%, more preferentially between 5% and 10% by weight relative to the total weight of the composition.

The composition of the invention also contains one or more solvents, which may be identical or different, chosen from polar (non-)protic organic solvents.

The term “organic solvent” is intended to mean an organic substance capable of dissolving another substance without chemically modifying it.

The organic solvents are preferably chosen from compounds that are liquid at a temperature of 25° C. and under 10⁵ Pa (760 mmHg).

The term “(non-)protic” is intended to mean a polar non-protic or polar protic solvent.

For the purposes of the invention, the term “polar solvent” is intended to mean a solvent constituted of molecules which have a dipolar moment. In particular, the polar solvents according to the invention preferably comprise at least one heteroatom such as oxygen.

The term “polar protic solvents” is intended to mean a polar solvent which also has at least one hydrogen atom capable of being involved in hydrogen bonds. In particular, the protic solvents according to the invention have at least one hydrogen atom bonded to a heteroatom.

The term “polar non-protic solvent” is intended to mean a polar solvent which does not have a hydrogen atom capable of being involved in hydrogen bonds.

Preferably, the polar (non-)protic organic solvent(s) is (are) chosen from:

(b1) monoalcohols comprising a hydroxyl group and a C₁-C₆(alkyl), preferably C₁-C₄(alkyl), group, such as methyl, ethyl or (iso)propyl,

(b2) polyols comprising from 2 to 30 hydroxyl groups and a C₂-C₈(alkyl) group, preferably a C₂-C₄(alkyl) group;

(b3) compounds comprising an —S(O)_{p′}— group with p equal to 1 or 2.

The polar (non-)protic organic solvent(s) can represent a total content ranging from 1% to 70% by weight, preferably from 2% to 60% by weight, better still from 5% to 50% by weight relative to the total weight of the composition.

The term “monoalcohol” is intended to mean an organic compound comprising a single “hydroxyl” (—OH) group; the organic compound may be aliphatic, acyclic, linear or branched.

Advantageously, said monoalcohol(s) comprise(s) from 2 to 4 carbon atoms.

Preferably, said monoalcohol(s) is (are) chosen from ethanol, propanol, isopropanol, butanol, and a mixture of these compounds, and preferably said monoalcohol(s) is (are) chosen from ethanol, isopropanol, and a mixture of these compounds, and more preferentially said monoalcohol is ethanol.

When they are present in the composition, the monoalcohols are in an amount of inclusively between 1% and 60%, preferably between 10% and 50%, more preferentially between 20% and 50% by weight relative to the total weight of the composition.

The term “polyols” is intended to mean an organic compound comprising at least two hydroxyl (—OH) groups, borne by different carbon atoms, this compound possibly being aliphatic, acyclic, linear or branched.

More particularly, the polyol(s) that can be used according to the invention comprise(s) from 2 to 30 hydroxyl groups, more preferentially from 2 to 10 hydroxyl groups, even more preferentially from 2 to 3 hydroxyl groups.

The polyol(s) that can be used according to the invention generally comprise(s) at least three carbon atoms.

Preferably, said polyol(s) that can be used according to the invention is (are) chosen from polyols comprising at least three carbon atoms and ethylene glycol, and preferably from propylene glycol, 1,3-propanediol, 1,3-butylene glycol, 1,2-pentanediol, dipropylene glycol, hexylene glycol, pentylene glycol, glycerol, ethylene glycol, and a mixture of these compounds, and more preferentially from propylene glycol, 1,3-propanediol, and a mixture of these compounds.

The solvent can be chosen from the compounds comprising an —S(O)_{p′}— group with p equal to 1 or 2.

In particular, the compound comprising an —S(O)_{p′}— group can be chosen from sulfones.

According to one particular embodiment of the invention, the solvent is chosen from the sulfones of formula (III)

Rₐ—S(O)ₚ—R′ₐ

In which formula (III):
Rₐ and R′ₐ, which may be identical or different, represent:
   a linear or branched (C₁-C₆)alkyl group, optionally substituted with one or more atoms or groups, which may be identical or different, chosen from i) halogen such as chlorine, ii) hydroxyl, iii) phenyl optionally substituted with one or more halogen, hydroxyl or (di)(C₁-C₄)(alkyl)amino atoms or groups;
   a phenyl group optionally substituted with one or more atoms or groups, which may be identical or different, chosen from i) halogen such as chlorine, ii) hydroxyl, iii) phenyl optionally substituted with one or more halogen, hydroxyl or (di)(C₁-C₄)(alkyl)amino atoms or groups, or else Rₐ and R′ₐ form, together with the sulfur atom of the sulfone group, a saturated or unsaturated, 4- or 6-membered heterocycle, preferably Rₐ and R′ₐ together form a divalent (C₃-C₆)alkylene or (C₃-C₆)alkenylene chain; and
p is 1 or 2, preferably 2.

Preferably, the sulfone is chosen from dimethyl sulfone, methyl ethyl ketone sulfone, diethyl sulfone, isopropyl methyl sulfone, isopropyl ethyl sulfone, diisopropyl sulfone, 2-chloroethyl ethyl sulfone, di-n-butyl sulfone, divinyl sulfone; diphenyl sulfone, bis(2-hydroxyethyl)sulfone, bis(4-hydroxyphenyl)sulfone, bis(4-aminophenyl)sulfone, bis(3-aminophenylsulfone), bis(4-chlorophenylsulfone), bis(4-fluorophenyl)sulfone, 2-hydroxyphenyl-4-hydroxyphenyl sulfone, phenyl-4-chlorophenyl sulfone, phenyl-2-aminophenyl sulfone, bis(3-amino-4-hydroxyphenyl)sulfone, dibenzyl sulfone; sulfolane and 3-sulfolene, preferably from dimethyl sulfone, bis(2-hydroxyethyl)sulfone, diethyl sulfone and methyl ethyl sulfone.

The compound comprising an —S(O)_{p′}— group can as a variant be chosen from sulfoxides.

Preferably, the solvent is chosen from the compounds of formula (III) such that p is 1 and preferably Rₐ and R′ₐ, which may be identical or different, represent a linear or branched (C₁-C₆)alkyl group, such as dimethyl sulfoxide, methylethyl sulfoxide or diethyl sulfoxide, it is preferably dimethylsulfoxide.

According to one particular embodiment of the invention, the solvents are chosen from polar protic solvents. Mention may in particular be made of the monoalcohols and the polyols as defined previously, and the sulfones of formula (III) in which at least one of the radicals Rₐ or R′ₐ comprises an OH, NH or NH₂ group.

According to another particular embodiment of the invention, the solvents are chosen from polar non-protic solvents. Mention may in particular be made of the sulfones of formula (III) in which neither Rₐ nor R′ₐ comprises an OH, NH or NH₂ group.

When it (they) is (are) present in the composition, the polyols and the compounds comprising an —S(O)ₚ— group are in an amount of inclusively between 1% and 50%, preferably between 5% and 30%, more preferentially between 5% and 15% by weight relative to the total weight of the composition.

According to one particularly preferred embodiment, the composition comprises at least two different polar (non-)protic solvents, in particular chosen from the monoalcohols, the polyols and the compounds comprising an —S(O)_{p′}— group.

More preferably, the composition comprises at least two different solvents each chosen from a different category (b1), (b2) and (b3).

In one preferred variant of the invention, the composition comprises at least one monoalcohol (b1) and at least one polar (non-)protic organic solvent chosen from one of categories (b2) and/or (b3). In one even more preferred variant, the composition comprises at least one monoalcohol (b1) and at least one polar (non-)protic organic solvent chosen from category (b2).

According to one particular embodiment of the invention, the composition does not comprise a thiol-comprising reducing agent such as thiolactic acid, preferably the composition does not comprise any reducing agent.

According to one embodiment of the invention, the composition does not comprise any alkaline agent.

Preferentially, the composition according to the invention is aqueous.

Preferentially, the composition according to the invention comprises water at a concentration ranging from 10% to 99.5%, better still from 30% to 95% and even better still from 40% to 90% by weight relative to the total weight of the composition.

The pH of the composition of the invention is less than or equal to 7. Preferably, the pH of the composition ranges from 1 to 6 and more preferably from 2 to 6.

The pH values may be adjusted with an organic or mineral acid, or with an alkaline agent chosen from mineral or organic or hybrid alkaline agents or mixtures thereof.

It is understood that the organic acid is different from the polar protic solvents.

The term “organic acid” is intended to mean an acid, i.e. a compound that is capable of releasing a cation or proton H⁺ or H₃O⁺, in aqueous medium, which comprises at least one optionally unsaturated, linear or branched C₁-C₂₀ hydrocarbon-based chain, a (hetero)cycloalkyl or (hetero)aryl group and at least one acidic chemical function chosen in particular from carboxyl C(O)OH, sulfuric SO₃H, SO₂H, and phosphoric PO₃H₂, PO₄H₂.

More particularly, the acids used are chosen from hydrochloric acid HCl, hydrobromic acid HBr, sulfuric acid H₂SO₄, alkylsulfonic acids: (C₁-C₆)Alk-S(O)₂OH such as methanesulfonic acid and ethanesulfonic acid; arylsulfonic acids: Ar—S(O)₂OH such as benzenesulfonic acid and toluenesulfonic acid; (C₁-C₆)alkoxysulfinic acids: Alk-O—S(O)OH such as methoxysulfinic acid and ethoxysulfinic acid; aryloxysulfinic acids such as tolueneoxysulfinic acid and phenoxysulfinic acid; phosphoric acid H₃PO₄; triflic acid CF₃SO₃H and tetrafluoroboric acid HBF₄, and carboxylic acid(s) of formula (K) below:

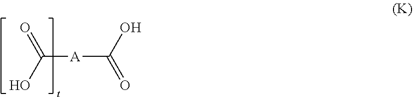

in which formula (K) or a salt thereof:
A represents a saturated or unsaturated, cyclic or non-cyclic, and aromatic or non-aromatic hydrocarbon-based group, which is monovalent when t is 0 or polyvalent when t is greater than or equal to 1, comprising from 1 to 50 carbon atoms, which is optionally interrupted with one or more heteroatoms and/or optionally substituted, especially with one or more hydroxyl groups; preferably, A represents a monovalent (C₁-C₆)alkyl group or a polyvalent (C₁-C₆)alkylene group optionally substituted with one or more hydroxyl groups.

Particularly, the carboxylic acid(s) of formula (K) as defined previously, and preferably the acid(s) used, is/are an alpha-hydroxy acid such as lactic acids, glycolic acids, tartaric acids or citric acids.

The mineral alkaline agent(s) are preferably chosen from aqueous ammonia, alkaline carbonates or bicarbonates such as sodium or potassium carbonates and sodium or potassium bicarbonates, sodium hydroxide or potassium hydroxide, or mixtures thereof.

According to an advantageous embodiment of the invention, the alkaline agent(s) are organic amines, i.e. they contain at least one substituted or unsubstituted amino group.

The organic alkaline agent(s) are more preferentially chosen from organic amines with a pK_{b} at 25° C. of less than 12, preferably of less than 10 and even more advantageously of less than 6. It should be noted that it concerns the pK_{b} corresponding to the function having the highest basicity.

Hybrid compounds that may be mentioned include the salts of the amines mentioned previously with acids such as carbonic acid or hydrochloric acid. The organic alkaline agent(s) are chosen, for example, from alkanolamines, oxyethylenated and/or oxypropylenated ethylenediamines, amino acids and the compounds of formula (L) below:

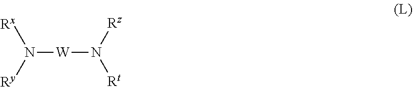

in which formula (L):
W is a divalent C₁-C₆ alkylene radical optionally substituted with a hydroxyl group or a C₁-C₆ alkyl radical, and/or optionally interrupted with one or more heteroatoms such as oxygen or NR^{u};
R^{x}, R^{y}, R^{z}, R^{t} and R^{u}, which may be identical or different, represent a hydrogen atom or a C₁-C₆ alkyl, C₁-C₆ hydroxyalkyl or C₁-C₆ aminoalkyl radical.

Preferably, the alkanolamine is ethanolamine (or monoethanolamine).

In one variant of the invention, the composition comprises, as alkaline agent, one or more alkanolamines (preferably ethanolamine) and aqueous ammonia. In this variant, the alkanolamine(s) are present in a predominant amount relative to the aqueous ammonia.

The composition of the invention can also comprise at least one usual cosmetic ingredient, in particular chosen from surfactants, in particular non-ionic or cationic surfactants, solid or liquid fatty substances, thickeners, in particular polysaccharide thickeners, cationic polymers, silicones, sunscreens; moisturizers; antidandruff agents; antioxidants; chelating agents; pearlescent agents and opacifiers; plasticizers and coalescence agents; fillers; fragrances; silanes; and crosslinking agents. The composition can, of course, comprise several cosmetic ingredients appearing in the above list.

Depending on their nature and the purpose of the composition, the usual cosmetic ingredients may be present in usual amounts, which can be readily determined by those skilled in the art and which may be, for each ingredient, between 0.01% and 80% by weight. Those skilled in the art will take care to select the ingredients included in the composition, and also the amounts thereof, so that they do not harm the properties of the compositions of the present invention.

The compositions used in the process according to the invention may be in any formulation form conventionally used, and especially in the form of an aqueous, alcoholic or aqueous-alcoholic, or oily, solution or suspension; a solution or a dispersion of the lotion or serum type; an emulsion, in particular of liquid or semi-liquid consistency, of the O/W, W/O or multiple type; a suspension or emulsion of soft consistency of cream (O/W) or (W/O) type; an aqueous or anhydrous gel, or any other cosmetic form.

These compositions may be packaged in pump-action bottles or in aerosol containers, so as to apply the composition in vaporized (lacquer) form or in the form of a mousse. Such packaging forms are indicated, for example, when it is desired to obtain a spray or a mousse, for treating the hair. In these cases, the composition preferably comprises at least one propellant.

The composition which has just been described can be applied to the hair. The bath ratio for the composition applied, that is to say the amount by weight of composition applied relative to the weight of hair treated, can range from 0.1 to 10, more particularly from 0.2 to 5, and preferably is between 0.5 and 3. The term “bath ratio” is intended to mean the ratio between the total weight of the applied composition and the total weight of keratin fibres to be treated.

The composition of the invention may be applied to dry or wet keratin materials, preferably to dry or wet hair, preferably to dry hair.

In particular, the step of applying the composition may be followed by a leave-on time. The leave-on time, namely the time of contact of the composition on the hair, is preferably at least 5 minutes, preferably between 10 and 60 minutes and preferably between 15 and 45 minutes. Rinsing of the hair may optionally be envisaged after the application of the composition and optionally the leave-on time.

The hair may then optionally be wrung dry, preferably wrung dry.

As previously indicated, the step of applying the composition which has been described is followed by a hair shaping step.

The shaping step can be a straightening/relaxing step or else a step for combing the hair.

This shaping step can be carried out by means of curlers, a curling iron or a straightening iron (also called flat tongs).

When the shaping step is carried out by means of an iron, it can be carried out at a temperature of at least 100° C.

Preferably, the iron is used at a temperature of at least 100° C., preferably at a temperature between, limits included, 100° C. and 300° C., preferably between 120° C. and 280° C., more preferably between 150° C. and 250° C., and better still between 200 and 250° C.

According to one particular embodiment, a straightening iron is used. In this embodiment, the straightening with the straightening iron is performed in several passes on the hair, in general 3 to 10 passes.

According to a particular embodiment, the process of the invention comprising the steps of applying the composition according to the invention to the hair, then of straightening with an iron, is performed one or more times, optionally separated by one or more cosmetic treatments, preferably a shampooing operation, until the desired shape or shape intensity is obtained.

According to one particular embodiment of the invention, a composition comprising an oxidizing agent can be applied to the hair, after the shaping step. This oxidation step can be followed by rinsing of the hair.

According to a particular embodiment of the invention, the process for treating keratin materials of the invention does not include any thiol-based reducing agent such as thiolactic acid; preferably, the process does not include any reducing agent.

According to a particular embodiment, the process for treating keratin materials does not include a lanthionization step. Preferably, the process for treating keratin fibres does not include an alkaline agent.

The examples that follow serve to illustrate the invention without, however, being limiting in nature.

### EXAMPLES

The following compositions were prepared from the ingredients indicated in the tables below, all the amounts being indicated as percent by weight of active material, relative to the total weight of the composition.

Examples: compositions T1 to T6 for comparative controls

| | T1 | T2 | T3 | T4 | T5 | T6 |
|---|---|---|---|---|---|---|
| Ethanol | | 30 | 30 | 30 | 30 | 30 |
| Butylene glycol | | | 10 | | | |
| 1,3-Propanediol | | | | 10 | | |
| Propylene glycol | | | | | 10 | |
| Dimethyl sulfone | | | | | | 10 |
| Water (qs) | 100 | 100 | 100 | 100 | 100 | 100 |

Examples: compositions 1 to 10 according to the invention

| | 1 | 2 | 3 | 4 | 5 |
|---|---|---|---|---|---|
| Thiolactone 38 | 10 | | | | |
| Thiolactone 107′ | | 10 | | | |
| Thiolactone 163 | | | 10 | | |
| Thiolactone 153 | | | | 10 | |
| Thiolactone 82 | | | | | 10 |
| Ethanol | 30 | 20 | 30 | 30 | 50 |
| Water (qs) | 100 | 100 | 100 | 100 | 100 |
| pH | 5.7 | 1.7 | 2.1 | 3.5 | 6.3 |

| | 6 | 7 | 8 |
|---|---|---|---|
| Thiolactone 38 | 5 | 5 | 5 |
| Ethanol | 30 | 30 | 30 |
| Butylene glycol | 10 | | |
| 1,3-Propanediol | | 10 | |
| Propylene glycol | | | 10 |
| Water (qs) | 100 | 100 | 100 |
| pH | 5.3 | 5.3 | 5.3 |

| | 9 | 10 |
|---|---|---|
| Thiolactone 38 | 10 | 15 |
| Thiolactone 82 | | |
| Ethanol | 30 | 30 |
| Dimethyl sulfone | 10 | 10 |
| Water (qs) | 100 | 100 |
| pH | 5.9 | 5.8 |

For each example, a lock of frizzy natural Caucasian hair of frizziness type IV, weighing 2.7 g having a length of 20 or 27 cm was treated in the following way:

The locks are wetted, a shampoo is applied (DOP camomille based on sodium laureth sulfate, on coco betaine, on glycerol and on cocamide MEA) in a proportion of 400 mg per lock. The locks are massaged and then rinsed with running water, wiped dry with a finger and dried with a hairdryer. The composition (from T1 to T6 for the controls, and from 1 to 10 according to the invention) is applied in a proportion of 2.7 g per lock, the lock is left in contact with the composition for 30 minutes, then it is dried with a hairdryer and straightened with flat tongs brought to 230° C. which are passed over each lock 10 times.

Each lock is then evaluated according to 3 main criteria for an indication of the performance level of the straightening, in the time:
I—Level of relaxation after 1 shampooing operation and natural drying
II—Level of relaxation after 5 shampooing cycles and natural drying
   The level of relaxation is evaluated using a set of reference locks, with the following grades:
      1: no relaxation
      2: medium relaxation
      3: good level of relaxation
      4: very good level of relaxation
         The results obtained for the controls are as follows:

Example

| | T1 | T2 | T3 | T4 | T5 | T6 |
|---|---|---|---|---|---|---|
| Criterion I | 1 | 1 | 1 | 2 | 2 | 1 |
| Criterion II | 1 | 1 | 1 | 1 | 1 | 1 |

The results obtained for the compositions according to the invention are as follows:

| | Example | | | | | | | | | |
|---|---|---|---|---|---|---|---|---|---|---|
| | 1 | 2 | 3 | 4 | 5 | 6 | 7 | 8 | 9 | 10 |
| Criterion I | 4 | 3 | 4 | 4 | 4 | 3 | 4 | 4 | 3 | 4 |
| Criterion II | 3 | 2 | 3 | 4 | 3 | 2 | 3 | 3 | 3 | 3 |

Moreover, locks having been bleached or dyed via a commercial oxidation colouring or relaxed locks are treated using the same protocol in order to evaluate the integrity and the latter is respected.

The composition according to the invention thus makes it possible to improve the relaxation of curls and the persistence thereof with respect to several shampooing operations, while at the same time keeping good integrity of the hair.

1.-25. (canceled)

26. A composition comprising:
(a) at least one compound chosen from the compounds of formula (I) below: 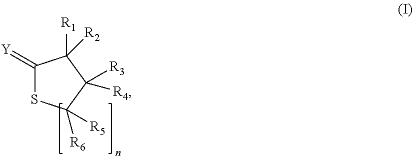
organic or mineral acid or base salts thereof, optical isomers thereof, geometrical isomers thereof, tautomers thereof, or solvates thereof;
wherein in formula (I):
Y represents an oxygen or sulfur atom or a group NR with R representing a hydrogen atom or a (C₁-C₆)alkyl or hydroxy(C₁-C₆)alkyl group;
R₁, R₂, R₃, R₄, R₅ and R₆, which may be identical or different, represent:
i) a hydrogen atom,
ii) hydroxyl,
iii) amino —NR′R″, with R′ and R″, which may be identical or different, representing a hydrogen atom or a (C₁-C₆)alkyl or hydroxy(C₁-C₆)alkyl group;
iv) cyano,
v) —(Y′)ₚ—C(Y″)—(Y′″)_{q}—R₇ with R₇ representing a hydrogen atom or a (C₁-C₆)alkyl group, p and q, which may be identical or different, being equal to 0 or 1, Y′, Y″ and Y′″, which may be identical or different, representing an oxygen or sulfur atom, or NR with R representing a hydrogen atom or a (C₁-C₆)alkyl or hydroxy(C₁-C₆)alkyl group,
vi) optionally substituted (C₁-C₆)alkyl,
vii) optionally substituted (C₂-C₆)alkenyl,
viii) optionally substituted (C₁-C₆)alkoxy,
or alternatively:
ix) R₁ and R₃ together form a covalent bond;
x) R₁ and R₂, and/or R₃ and R₄, and/or R₅ and R₆ form, together with the carbon atom that bears them, a double bond oxo ═O or ═C(R₉)—R₁₀ with R₉ and R₁₀ representing a) a hydrogen atom or a group chosen from b) hydroxyl, c) (C₁-C₆)alkyl optionally substituted with one or more hydroxyl groups, and d) (C₂-C₆)alkenyl, e) —(Y′)ₚ—C(Y″)—(Y′″)_{q}—R₇ with p and q, which may be identical or different, being equal to 0 or 1, Y′, Y″ and Y′″, which may be identical or different, representing an oxygen or sulfur atom, and R₇ representing a hydrogen atom or a (C₁-C₆)alkyl group;
n represents an integer between 0 and 6 inclusive;
wherein:
when n is greater than or equal to 2, then the contiguous groups C(R₅)—R₆ may be identical or different; and
(b) at least one solvent, which may be identical or different, chosen from polar (non-)protic organic solvents;
wherein the pH of the composition is less than or equal to 7.

27. The composition according to claim 26, wherein the at least one compound of formula (I) is such that n is 1 or 2 and the compound is selected from compounds of formula (Ia): 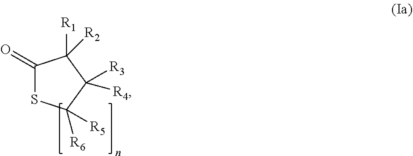
organic or mineral acid or base salts thereof, optical isomers thereof, geometrical isomers thereof, tautomers thereof, or solvates thereof;
wherein in formula (Ia):
R₁, R₂, R₃, R₄, R₅ and R₆, which may be identical or different, represent:
i) a hydrogen atom,
ii) hydroxyl,
iii) amino —NR′R″, with R′ and R″, which may be identical or different, representing a hydrogen atom or a (C₁-C₆)alkyl or hydroxy(C₁-C₆)alkyl group;
iv) cyano,
v) —(Y′)ₚ—C(O)—(Y″)_{q}—R₇ with R₇ representing a hydrogen atom or a (C₁-C₆)alkyl group, p and q, which may be identical or different, being equal to 0 or 1, with Y′ and Y″, which may be identical or different, representing an oxygen atom or NR with R representing a hydrogen atom or a (C₁-C₆)alkyl or hydroxy(C₁-C₆)alkyl group,
vi) (C₁-C₆)alkyl optionally substituted with one or more groups chosen from hydroxyl and —C(O)—R₈ with R₈ representing a) a hydrogen atom, b) a hydroxyl group, c) (C₁-C₆)alkoxy,
vii) (C₂-C₆)alkenyl,
viii) (C₁-C₆)alkoxy,
or alternatively:
ix) R₁ and R₂, and/or R₃ and R₄, and/or R₅ and R₆ form, together with the carbon atom that bears them, a double bond oxo ═O or ═C(R₉)—R₁₀ with R₉ and R₁₀ representing a) a hydrogen atom or a group chosen from b) hydroxyl, c) (C₁-C₆)alkyl.

28. The composition according to claim 26, wherein in the at least one compound of formula (I) n is 1, and the compound is selected from compounds of formula (I′a): 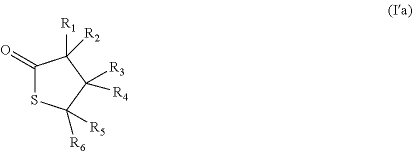
organic or mineral acid or base salts thereof, optical isomers thereof, geometrical isomers thereof, tautomers thereof, or solvates thereof;
wherein in formula (I′b):
R₁, R₂, R₃, R₄, R₅ and R₆, which may be identical or different, represent:
i) a hydrogen atom,
ii) hydroxyl,
iii) amino —NR′R″, with R′ and R″, which may be identical or different, representing a hydrogen atom or a (C₁-C₆)alkyl or hydroxy(C₁-C₆)alkyl group;
iv) cyano,
v) —(Y′)ₚ—C(O)—(O)_{q}—R₇ with R₇ representing a hydrogen atom or a (C₁-C₆)alkyl group, p and q, which may be identical or different, being equal to 0 or 1, with p+q=0 or 1, with Y′ representing an oxygen atom or NR with R representing a hydrogen atom or a (C₁-C₆)alkyl or hydroxy(C₁-C₆)alkyl group;
vi) optionally substituted (C₁-C₆)alkyl,
vii) (C₂-C₆)alkenyl,
viii) (C₁-C₆)alkoxy,
wherein the alkyl groups are optionally substituted with one or more groups chosen from hydroxyl and —(O)ₜ—C(O)—R₈ with t being equal to 0 or 1, R₈ representing a) a hydrogen atom, b) a hydroxyl group, c) (C₁-C₆)alkyl, d) (C₁-C₆)alkoxy, e) (di)(C₁-C₆)(alkyl)amino, f) (di)hydroxy(C₁-C₆)alkylamino;
or alternatively:
ix) R₁ and R₂, and/or R₃ and R₄, and/or R₅ and R₆ form, together with the carbon atom that bears them, a double bond oxo ═O or ═C(R₉)—R₁₀ with R₉ and R₁₀ representing i) a hydrogen atom or a group chosen from ii) hydroxyl, iii) (C₁-C₆)alkyl optionally substituted with one or more hydroxyl groups.

29. The composition according to claim 26, wherein in the at least one compound of formula (I) n is 1, and the compound is selected from compounds of formula (Ic): 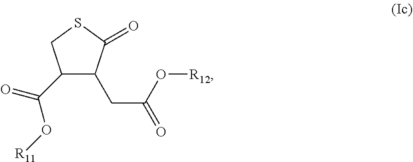
organic or mineral acid or base salts thereof, optical isomers thereof, geometrical isomers thereof, tautomers thereof, or solvates thereof;
wherein in formula (Ic):
R₁₁ and R₁₂, which may be identical or different, represent a hydrogen atom or a (C₁-C₆)alkyl group.

30. The composition according to claim 26, wherein in the at least one compound of formula (I) n is 1 or 2, and the compound is selected from the compounds of formula (Ib): 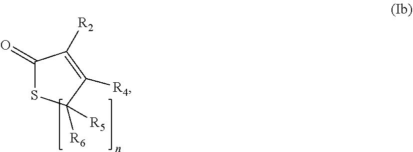
organic or mineral acid or base salts thereof, optical isomers thereof, geometrical isomers thereof, tautomers thereof, or solvates thereof;
wherein in formula (Ib):
R₂, R₄, R₅ and R₆, which may be identical or different, represent:
i) a hydrogen atom,
ii) hydroxyl,
iii) amino —NR′R″, R′ and R″, which may be identical or different, representing a hydrogen atom or a (C₁-C₆)alkyl or hydroxy(C₁-C₆)alkyl group;
iv) cyano,
v) —(Y′)ₚ—C(O)—(O)_{q}—R₇ with R₇ representing a hydrogen atom or a (C₁-C₆)alkyl group, p and q, which may be identical or different, being equal to 0 or 1, with p+q=0 or 1, with Y′ representing an oxygen atom or NR with R representing a hydrogen atom or a (C₁-C₆)alkyl or hydroxy(C₁-C₆)alkyl group,
vi) (C₁-C₆)alkyl optionally substituted with one or more groups chosen from hydroxyl and —C(O)—R₈ with R₈ representing a) a hydrogen atom, b) a hydroxyl group, c) (C₁-C₆)alkoxy,
vii) (C₂-C₆)alkenyl,
viii) (C₁-C₆)alkoxy,
or alternatively:
ix) R₅ and R₆ form, together with the carbon atom that bears them, a double bond oxo ═O or ═C(R₉)—R₁₀ with R₉ and R₁₀ representing a hydrogen atom or a (C₁-C₆)alkyl group.

31. The composition according to claim 26, wherein the at least one compound of formula (I) is chosen from the following compounds: 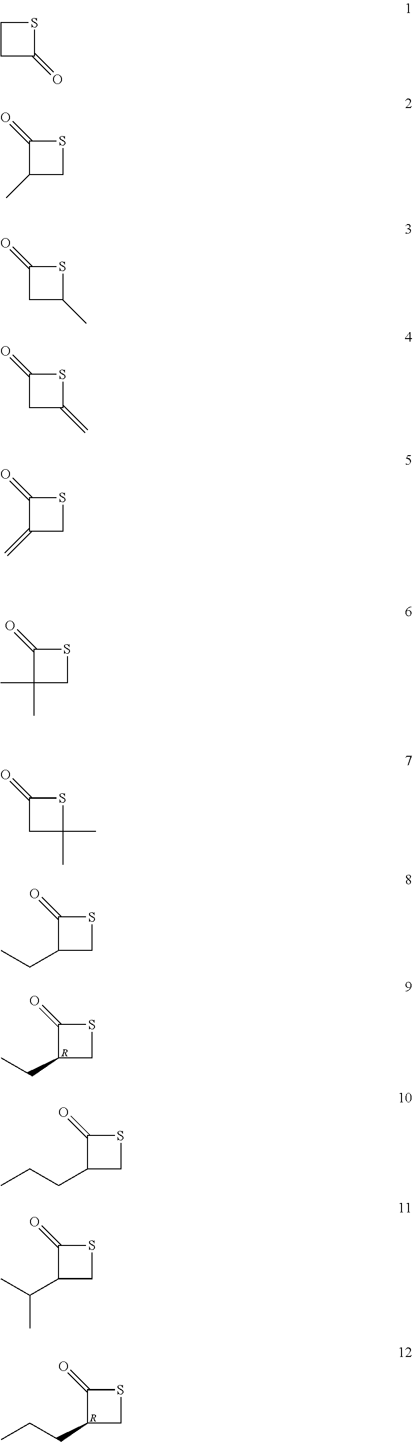 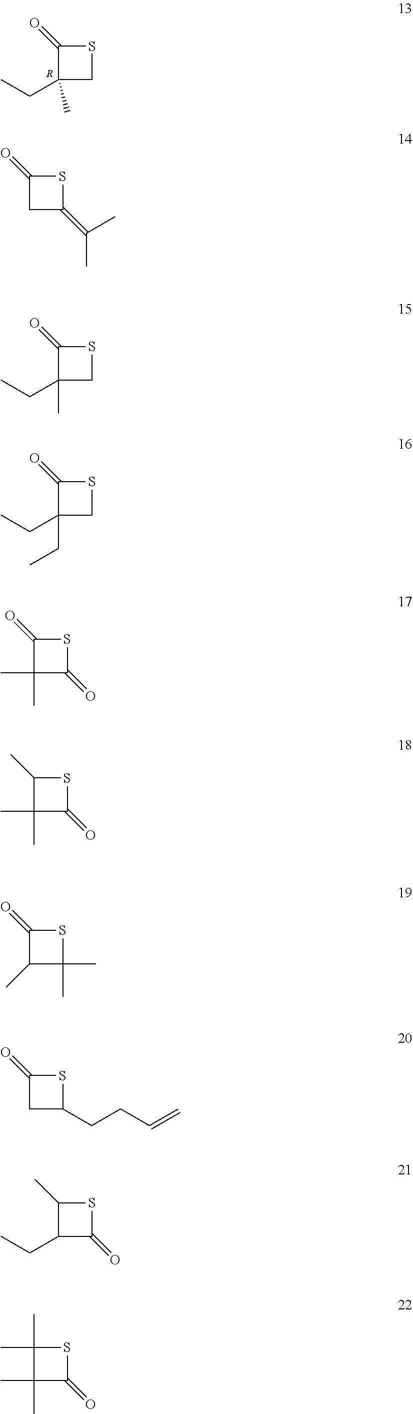 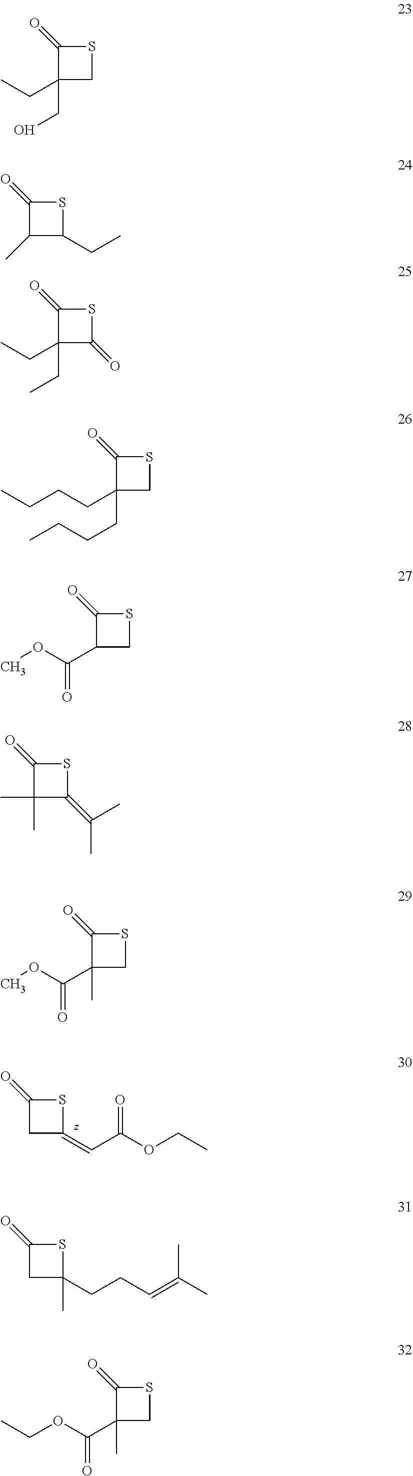 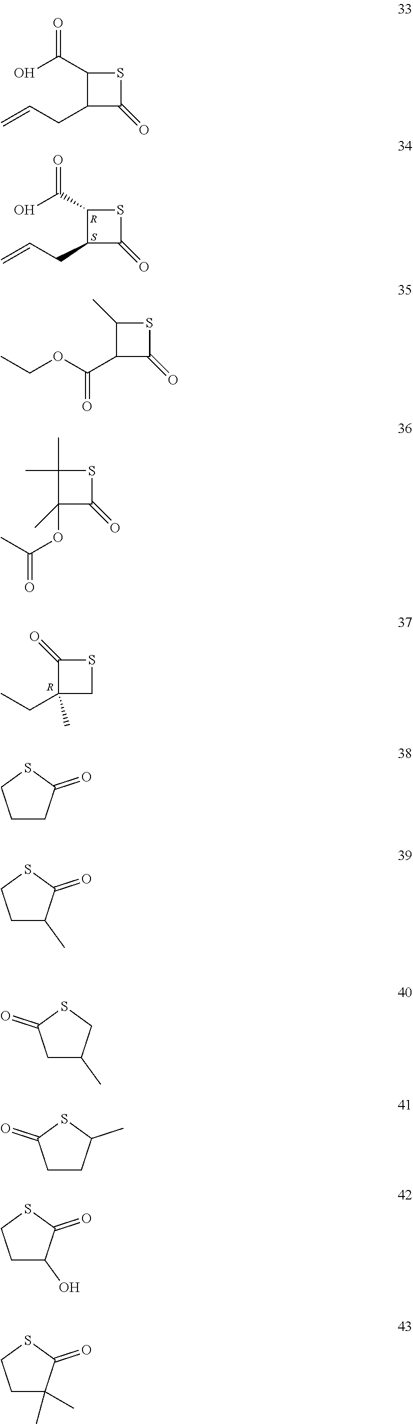 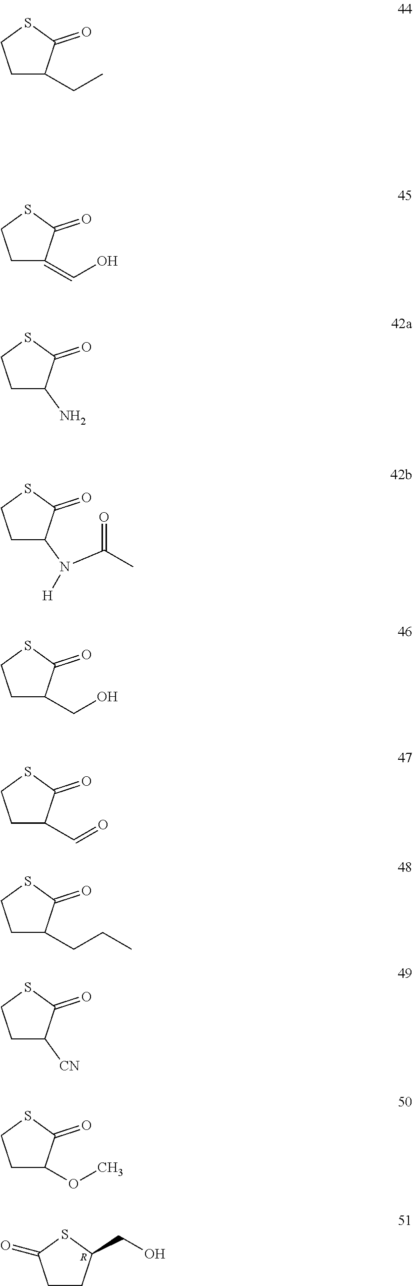 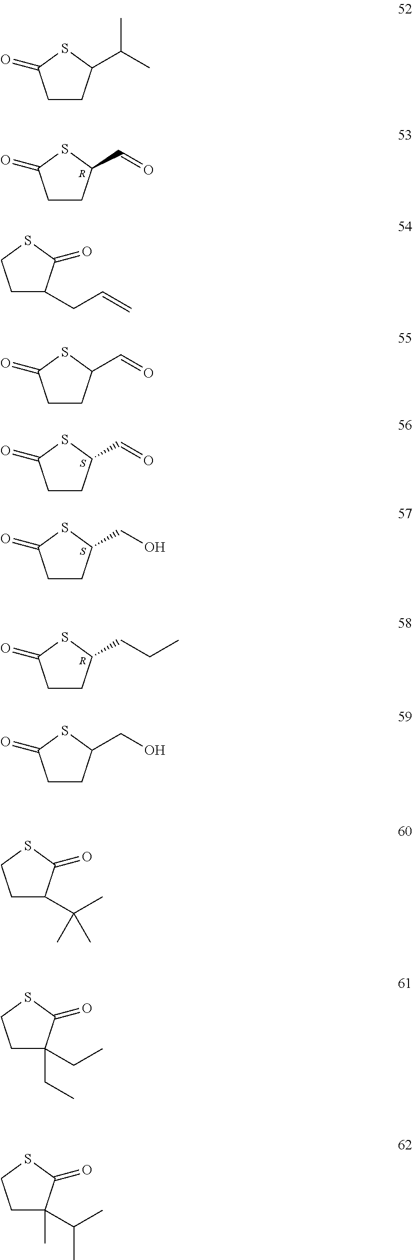 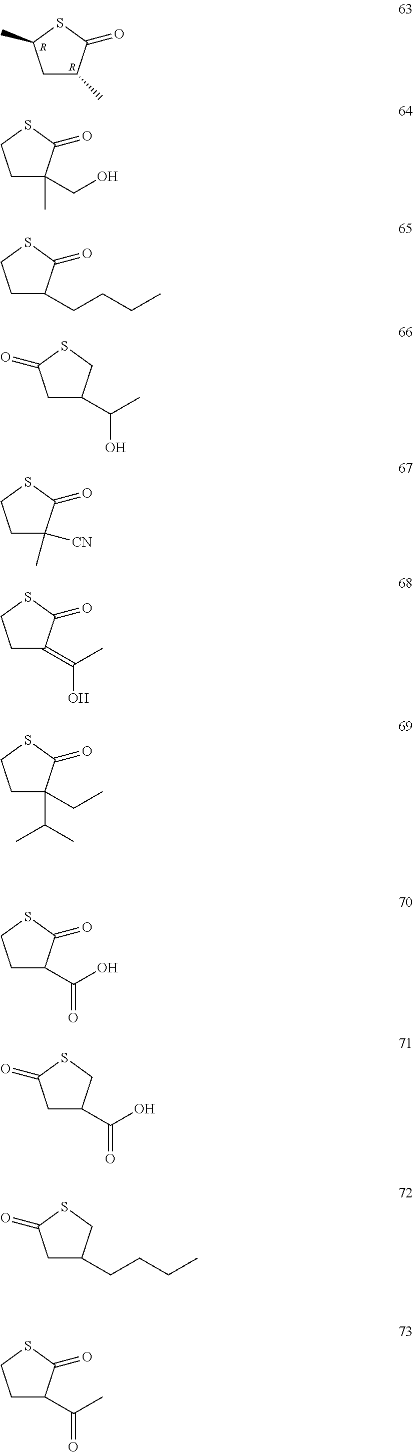 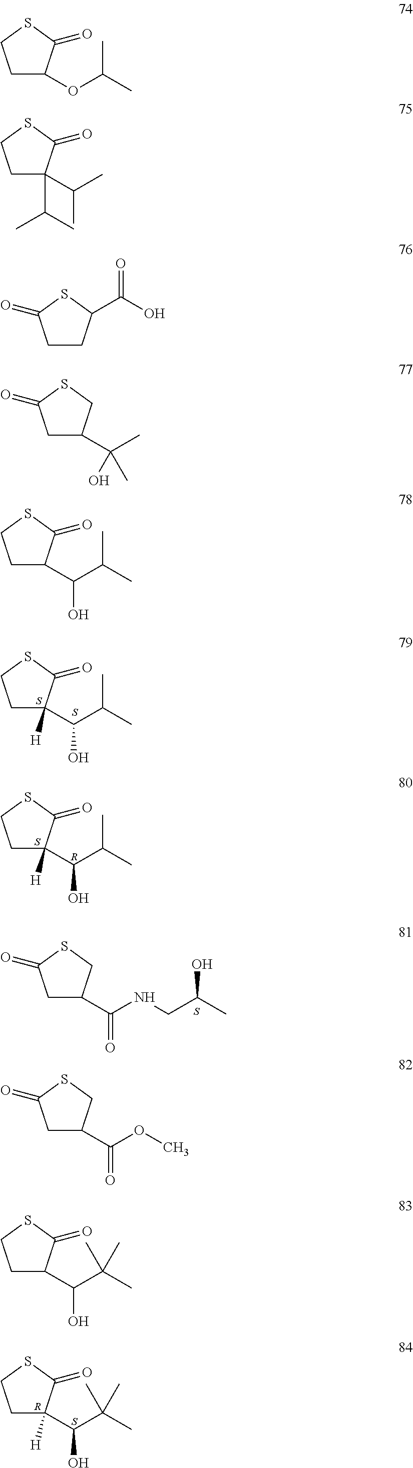 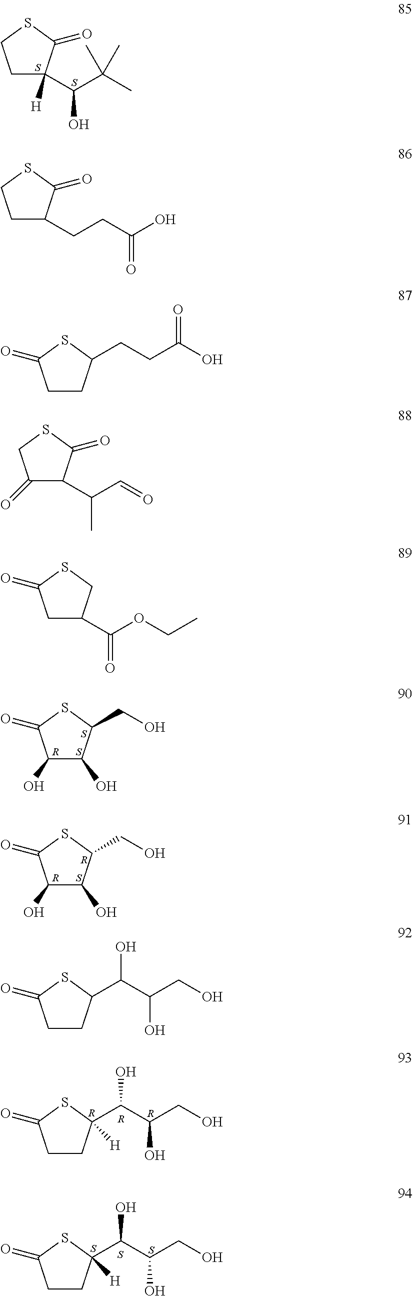 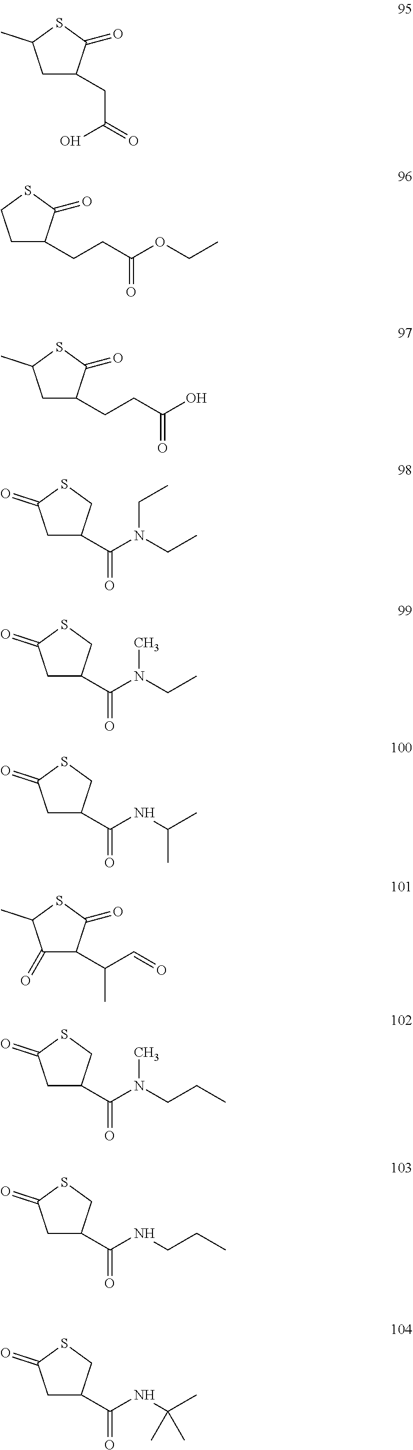 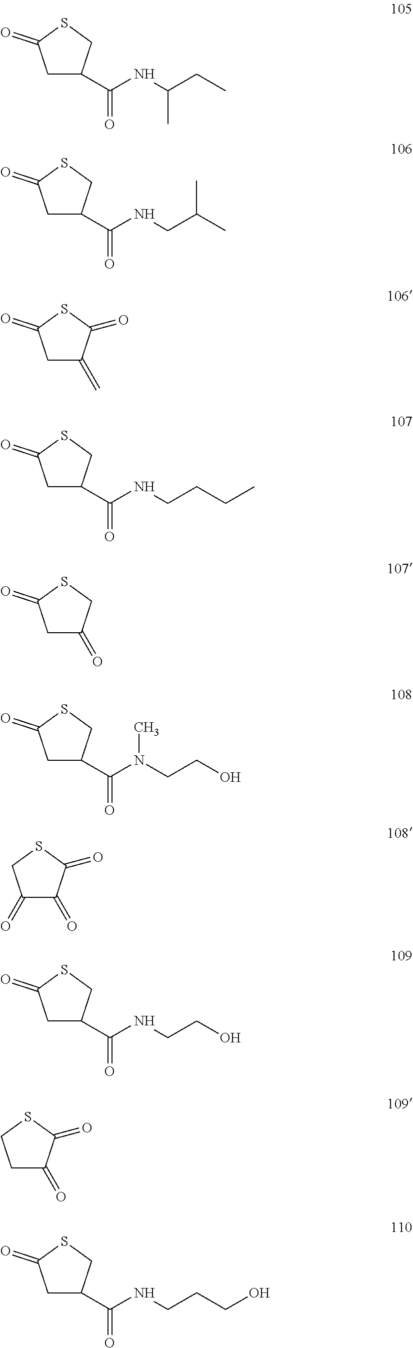 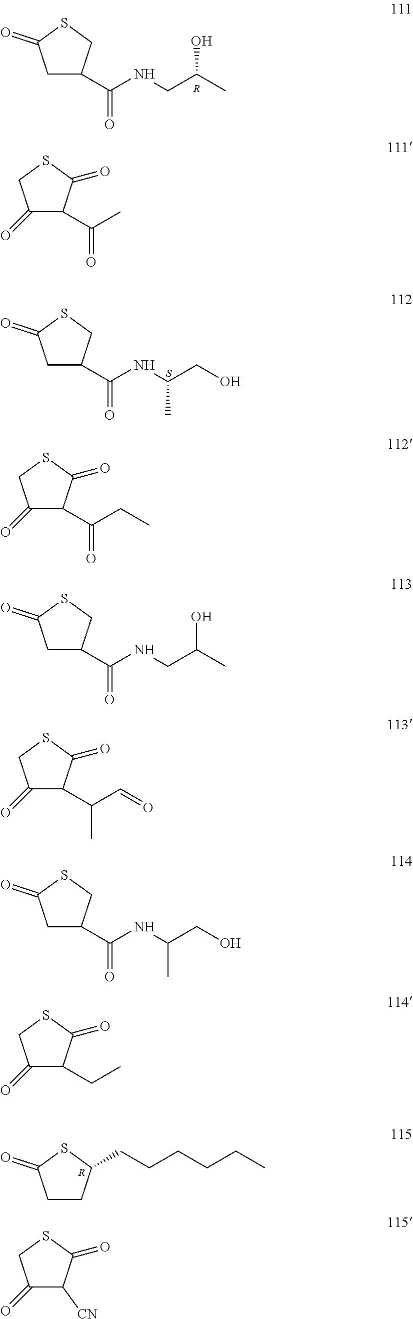 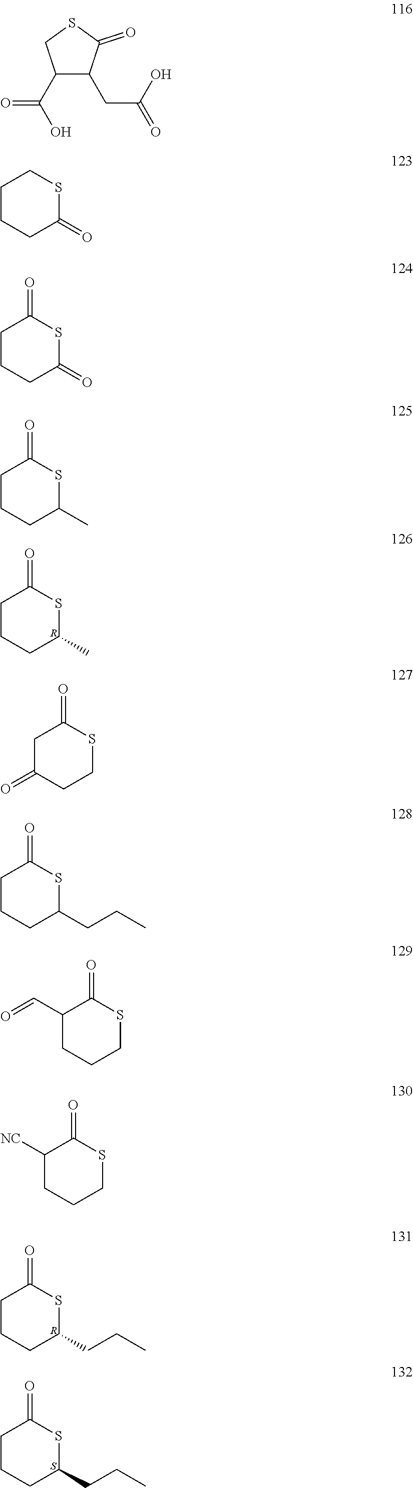 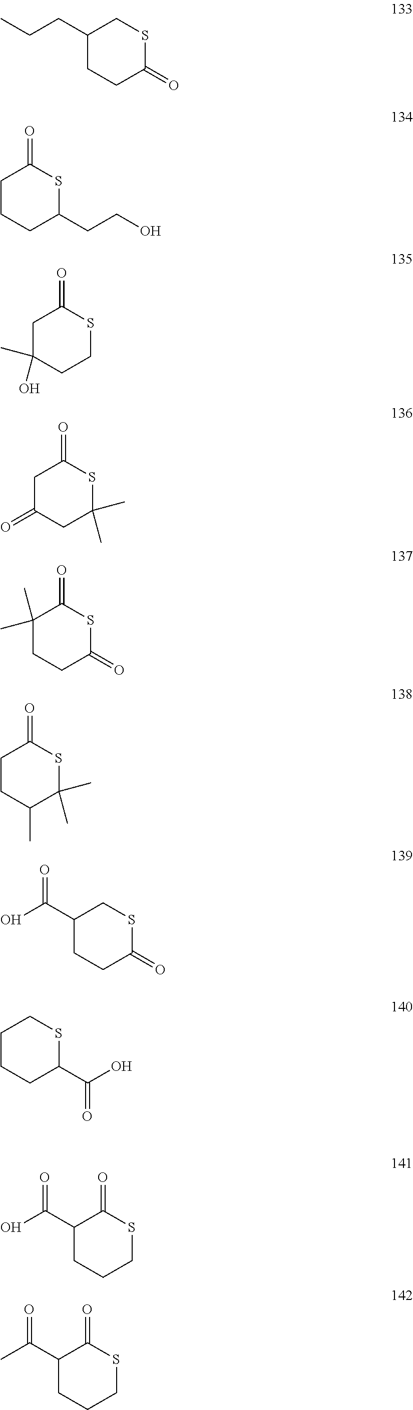 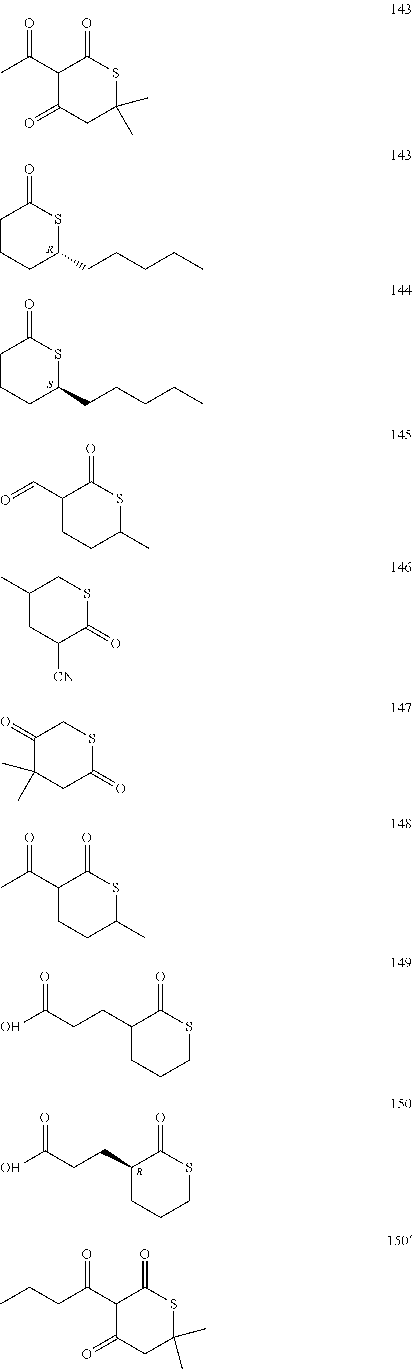 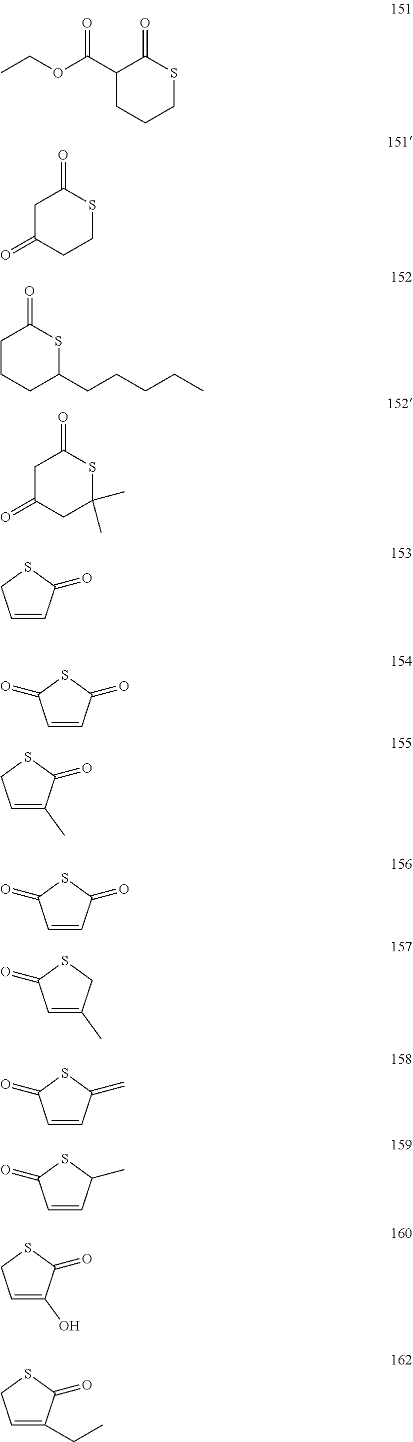 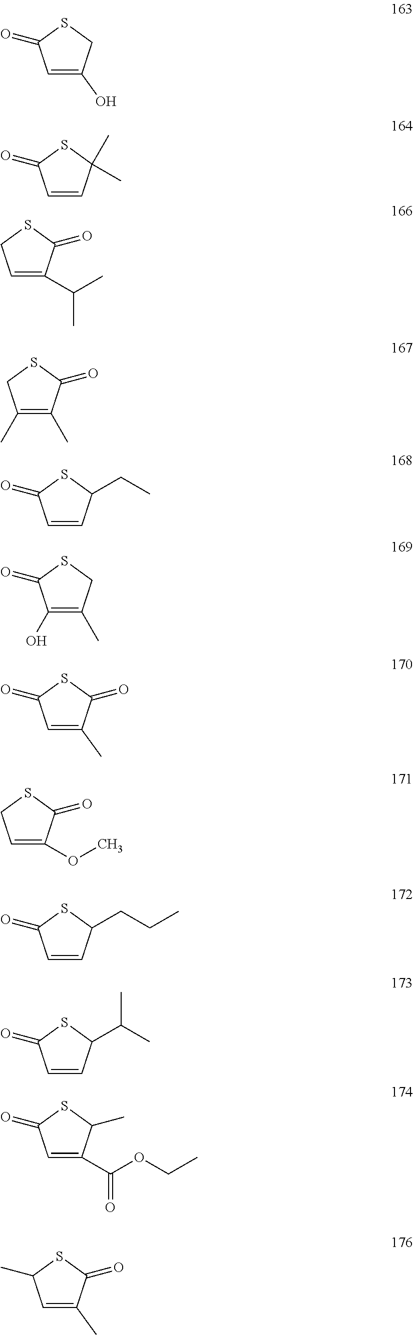 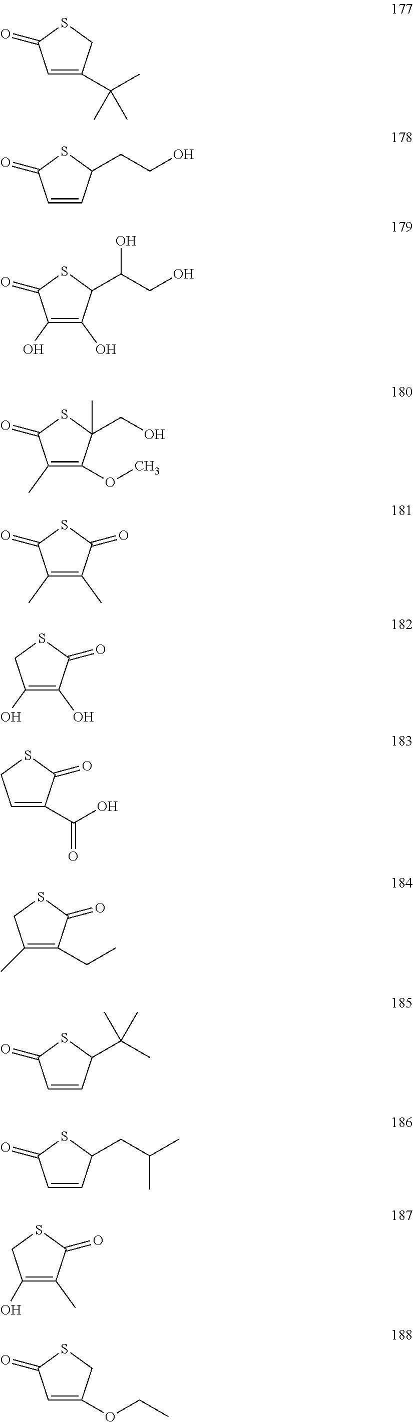 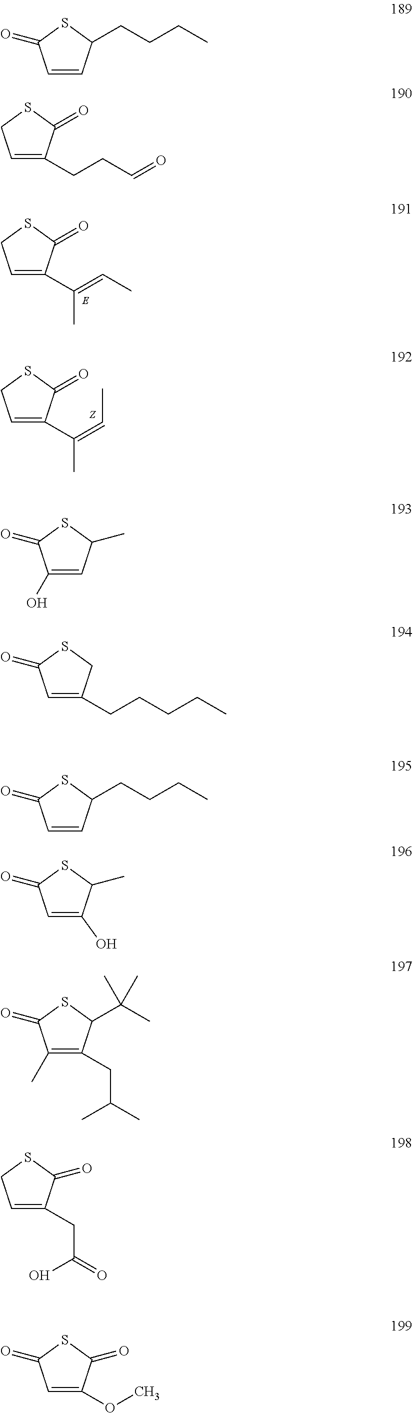 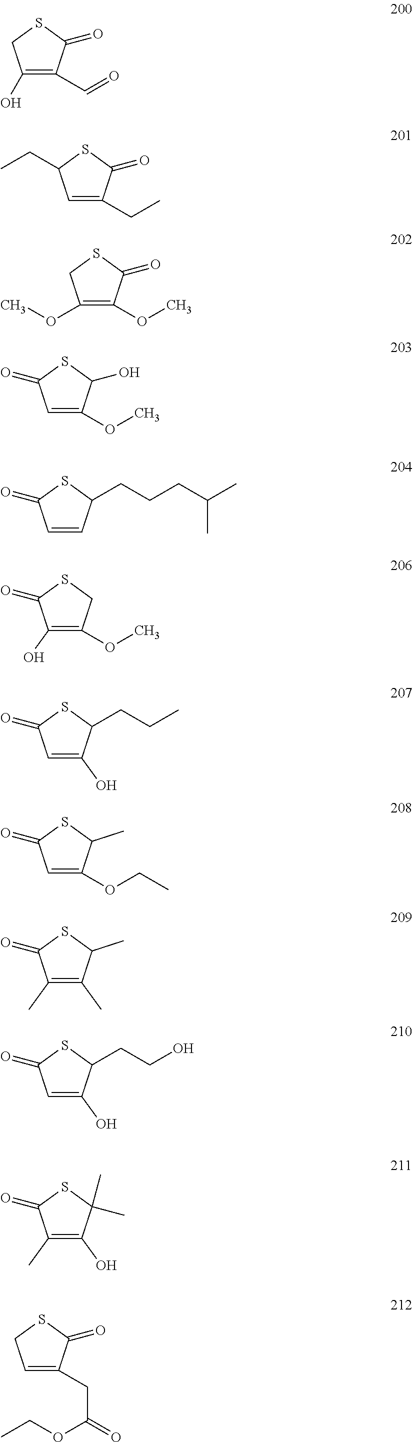 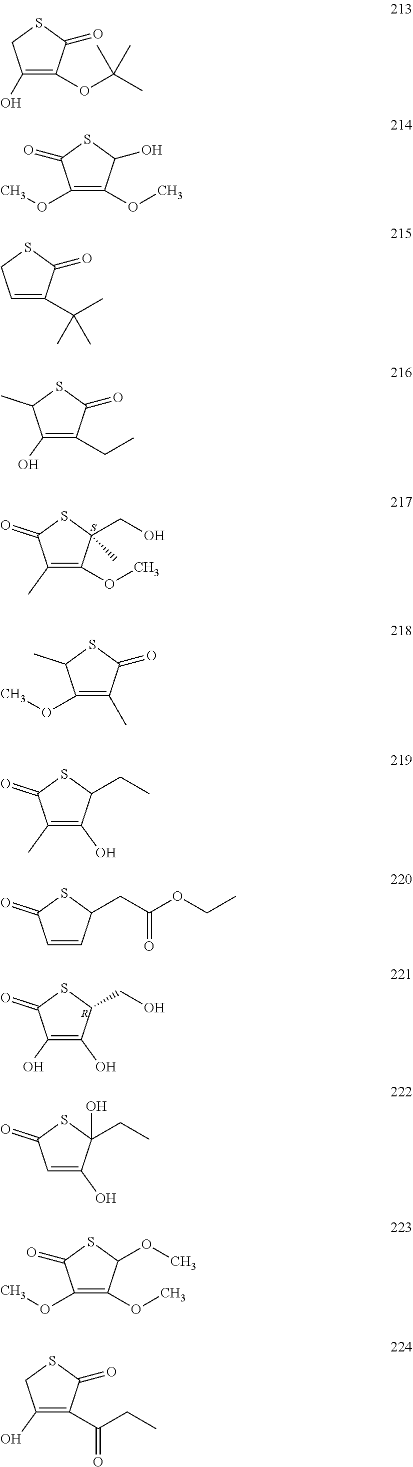 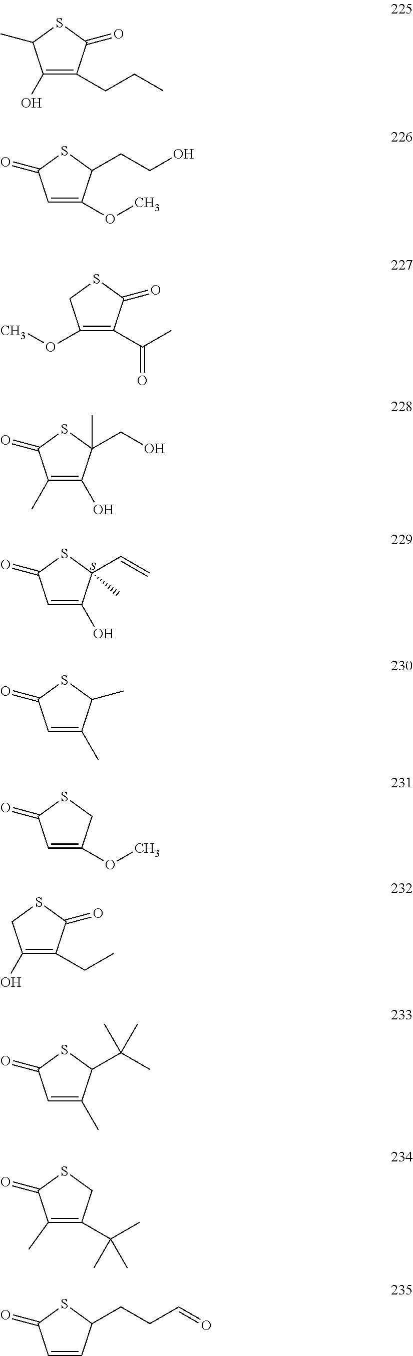 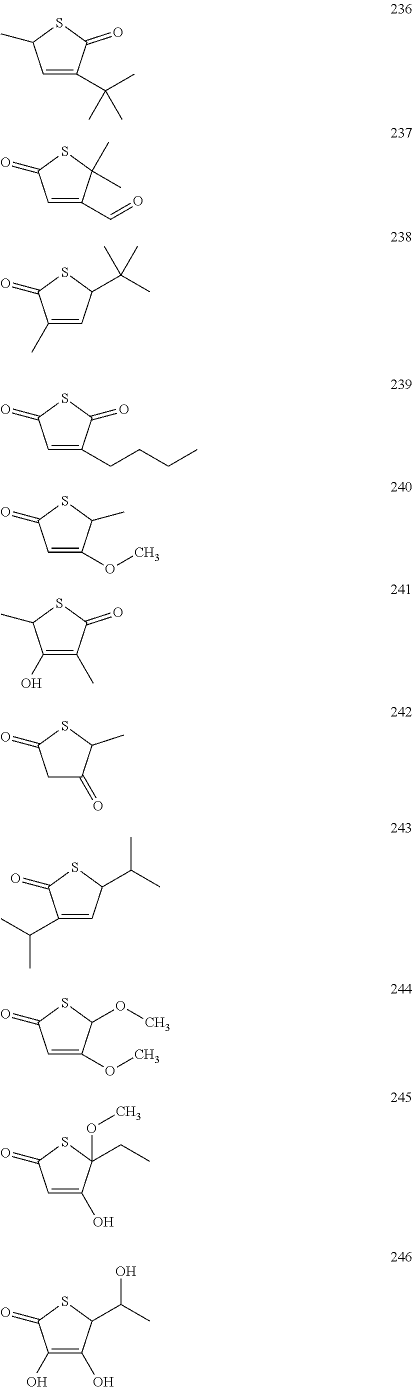 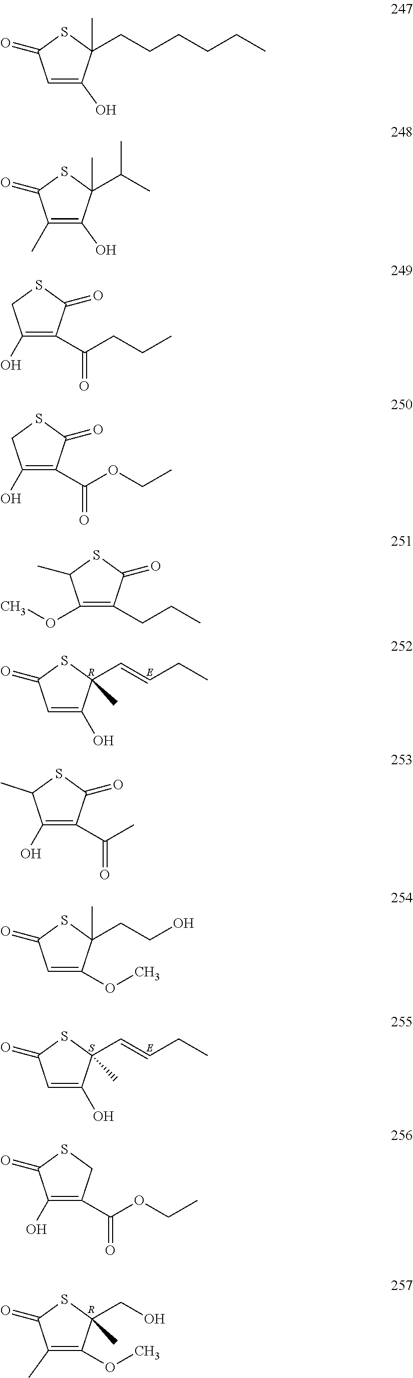 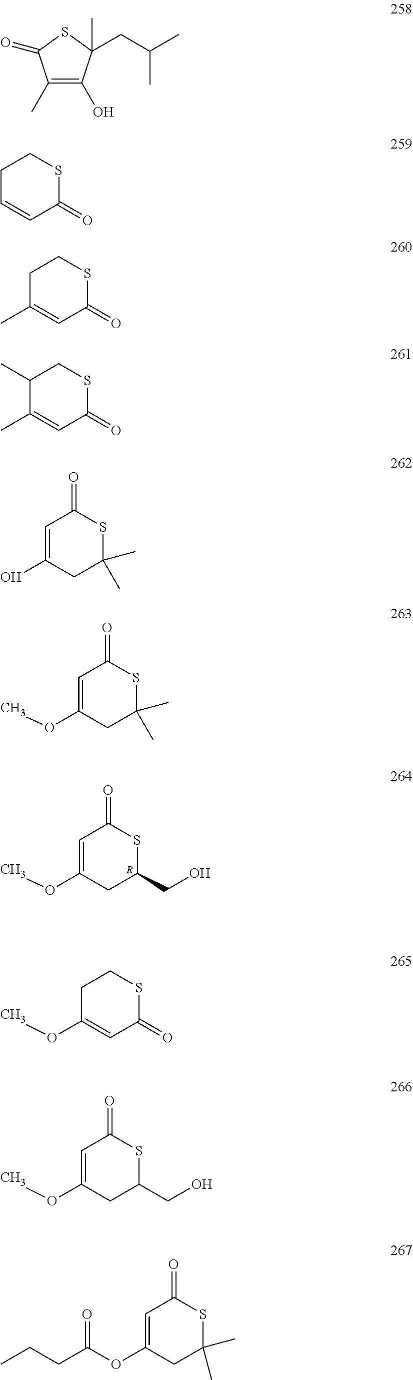
organic or mineral acid or base salts thereof, optical isomers thereof, geometrical isomers thereof, tautomers thereof, or solvates thereof.

32. The composition according to claim 31, wherein the at last one compound of formula (I) is chosen from the compounds of formulae (38) to (122) and (153) to (258).

33. The composition according to claim 26, wherein the at least one compound of formula (I) is present in an amount ranging from 0.01% to 50% by weight, relative to the total weight of the composition.

34. The composition according to claim 26, wherein the at least one polar (non-)protic organic solvent is chosen from:
(b1) monoalcohols comprising a hydroxyl group and a C₁-C₆(alkyl);
(b2) polyols comprising from 2 to 30 hydroxyl groups and a (C₂-C₈)alkyl group;
(b3) the compounds of formula Rₐ—S(O)ₚ—Rₐ′, wherein Rₐ and Rₐ′, which may be identical or different, represent a (C₁-C₆)alkyl, and p is equal to 1 or 2.

35. The composition according to claim 34, wherein the composition comprises at least two solvents each chosen from a different category (b1), (b2) and (b3).

36. The composition according to claim 34, wherein the monoalcohols are chosen from ethanol, propanol, isopropanol, butanol, or a mixture of these compounds.

37. The composition according to claim 34, wherein the polyalcohols are chosen from polyols comprising at least three carbon atoms and ethylene glycol.

38. The composition according to claim 34, wherein the solvents are chosen from the compounds of formula Rₐ—S(O)ₚ—Rₐ′ (III) below
wherein in formula (III):
Rₐ—S(O)ₚ—R′ₐ
Wherein Rₐ and R′ₐ, which may be identical or different, represent:
a linear or branched (C₁-C₆)alkyl group, optionally substituted with one or more atoms or groups, which may be identical or different, chosen from i) halogen, ii) hydroxyl, iii) phenyl optionally substituted with one or more halogen, hydroxyl or (di)(C₁-C₄)(alkyl)amino atoms or groups;
a phenyl group optionally substituted with at least one atom or group, which may be identical or different, chosen from i) halogen, ii) hydroxyl, iii) phenyl optionally substituted with one or more halogen, hydroxyl or (di)(C₁-C₄)(alkyl)amino atoms or groups,
or else Rₐ and R′ₐ form, together with the sulfur atom of the sulfone group, a saturated or unsaturated, 4- or 6-membered heterocycle, preferably Rₐ and R′ₐ together form a divalent (C₃-C₆)alkylene or (C₃-C₆)alkenylene chain; and
p is 1 or 2

39. The composition according to claim 34, wherein the solvents are chosen from the compounds of formula Rₐ—S(O)ₚ—Rₐ′ wherein p is 1 and preferably Rₐ and R′ₐ, which may be identical or different, represent a linear or branched (C₁-C₆)alkyl group.

40. The composition according to claim 26, wherein the at least one polar (non-)protic organic solvent is a polar protic organic solvent.

41. The composition according to claim 26, wherein the at least one polar (non-)protic solvent is present in a total amount ranging from 1% to 70% by weight, relative to the total weight of the composition.

42. The composition according to claim 34, wherein the at least one solvent (b1) is present in an amount ranging from 1% to 60% by weight, relative to the total weight of the composition.

43. The composition according to claim 34, wherein the at least one solvent (b2) or (b3) is present in an amount ranging from 1% to 50% by weight, relative to the total weight of the composition.

44. The composition according to claim 26, wherein the composition has a pH ranging from 1 to 6.

45. The composition according to claim 26, wherein the composition does not comprise any thiol-based reducing agents.

46. A process for treating keratin materials, comprising applying to the keratin materials a composition comprising:
(a) at least one compound chosen from the compounds of formula (I) below: 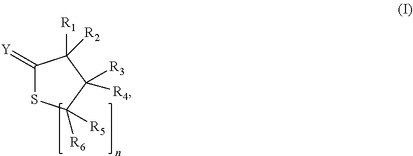
organic or mineral acid or base salts thereof, optical isomers thereof, geometrical isomers thereof, tautomers thereof, or solvates thereof;
wherein in formula (I):
Y represents an oxygen or sulfur atom or a group NR with R representing a hydrogen atom or a (C₁-C₆)alkyl or hydroxy(C₁-C₆)alkyl group;
R₁, R₂, R₃, R₄, R₅ and R₆, which may be identical or different, represent:
i) a hydrogen atom,
ii) hydroxyl,
iii) amino —NR′R″, with R′ and R″, which may be identical or different, representing a hydrogen atom or a (C₁-C₆)alkyl or hydroxy(C₁-C₆)alkyl group;
iv) cyano,
v) —(Y′)ₚ—C(Y″)—(Y′″)_{q}—R₇ with R₇ representing a hydrogen atom or a (C₁-C₆)alkyl group, p and q, which may be identical or different, being equal to 0 or 1, Y′, Y″ and Y′″, which may be identical or different, representing an oxygen or sulfur atom, or NR with R representing a hydrogen atom or a (C₁-C₆)alkyl or hydroxy(C₁-C₆)alkyl group,
vi) optionally substituted (C₁-C₆)alkyl,
vii) optionally substituted (C₂-C₆)alkenyl,
viii) optionally substituted (C₁-C₆)alkoxy,
or alternatively:
ix) R₁ and R₃ together form a covalent bond;
x) R₁ and R₂, and/or R₃ and R₄, and/or R₅ and R₆ form, together with the carbon atom that bears them, a double bond oxo ═O or ═C(R₉)—R₁₀ with R₉ and R₁₀ representing a) a hydrogen atom or a group chosen from b) hydroxyl, c) (C₁-C₆)alkyl optionally substituted with one or more hydroxyl groups, and d) (C₂-C₆)alkenyl, e) —(Y′)ₚ—C(Y″)—(Y′″)_{q}—R₇ with p and q, which may be identical or different, being equal to 0 or 1, Y′, Y″ and Y′″, which may be identical or different, representing an oxygen or sulfur atom, and R₇ representing a hydrogen atom or a (C₁-C₆)alkyl group;
n represents an integer between 0 and 6 inclusive;
wherein:
when n is greater than or equal to 2, then the contiguous groups C(R_{S})—R₆ may be identical or different; and
(b) at least one solvent, which may be identical or different, chosen from polar (non-)protic organic solvents;
wherein the pH of the composition is less than or equal to 7.

47. The process according to claim 46, wherein applying the composition to the hair is followed by shaping the hair by means of a heat treatment at a temperature of at least 100° C.

48. The process according to claim 46, wherein the process does not use any thiol-comprising reducing agent.
